(19) United States
(12) Patent Application Publication
(10) Pub. No.: US 2026/0202742 A1
(43) Pub. Date: Jul. 16, 2026

(54) PHOTOSENSITIVE RESIN COMPOSITION, RESIN FILM HAVING PATTERN AND METHOD FOR PRODUCING SAME, AND SEMICONDUCTOR CIRCUIT BOARD

(71) Applicant: JSR CORPORATION, Tokyo (JP)

(72) Inventors: Ryoji TATARA, Minato-ku, Tokyo (JP); Shin-Ya NAKAFUJI, Minato-ku, Tokyo (JP); Tetsuya SAKUMA, Minato-ku, Tokyo (JP); Ryuichi OKUDA, Minato-ku, Tokyo (JP); Takashi DOI, Minato-ku, Tokyo (JP); Junichi OOTO, Minato-ku, Tokyo (JP); Takahiro KATO, Minato-ku, Tokyo (JP)

(73) Assignee: JSR CORPORATION, Tokyo (JP)

(21) Appl. No.: 19/136,348

(22) PCT Filed: Dec. 5, 2023
(86) PCT No.: PCT/JP2023/043471

§ 371 Date: Jun. 6, 2025

(30) Foreign Application Priority Data
Dec. 8, 2022 (JP) 2022-196583

(51) Int. Cl. G03F 7/004; C07D 239/50; C07D 251/70; C07D 307/90; C08G 73/06; G03F 7/039; G03F 7/32; H10W 70/695

(52) U.S. Cl. CPC G03F 7/0045; C07D 239/50; C07D 251/70; C07D 307/90; C08G 73/0672; G03F 7/0395; G03F 7/322; H10W 70/695

(57) ABSTRACT

A photosensitive resin composition contains an alkali-soluble polymer (A), a photosensitizer (B), a crosslinking compound (C) having a crosslinkable group that reacts with a component (D) below by heat, a compound (D) represented by formula (1), and a solvent (E). X¹¹ represents, for example, a nitrogen atom, R¹¹ represents a hydrogen atom or a group represented by formula (2). L²¹ represents, for example, a single bond, R²¹ represents, for example, a hydrogen atom, R²² represents a monovalent organic group, and n²¹ to n²³ represent an integer of 0 or more. At least one of three R¹¹'s is represented by formula (2), and in at least one group represented by formula (2), n²¹ is an integer of 1 or more, and at least one R²¹ is a hydrogen atom.

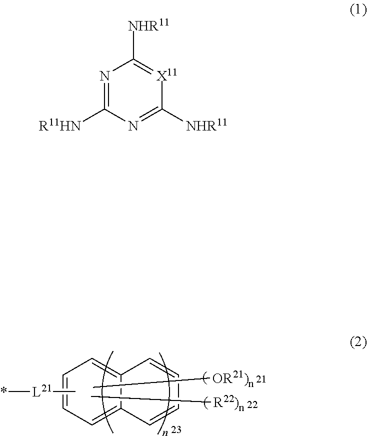

### TECHNICAL FIELD

The present invention relates to a photosensitive resin composition, a resin film having a pattern and a method for producing the same, and a semiconductor circuit board.

### BACKGROUND ART

The present invention relates to a photosensitive resin composition suitably used for forming, for example, a surface protective film and an interlayer insulation film included in, for example, an electronic component, and further relates to a cured film formed from the composition, a method for producing the cured film, and an electronic component including the cured film.

Conventionally, various photosensitive resin compositions have been proposed as materials used for forming, for example, a surface protective film and an interlayer insulation film used for a semiconductor circuit board in an electronic component. For example, a photosensitive resin composition containing a resin having a phenolic hydroxy group as an alkali-soluble resin has been studied (Patent Literatures 1 and 2).

Further, a package technology using a silicon interposer or a fan-out type package technology using a mold substrate, for example, has been proposed to increase a density and performance of a semiconductor circuit board. In order to ensure the reliability of such a semiconductor package, adhesion of an insulation film to a substrate is an important factor, and an adhesion aid and other additives contained in the photosensitive resin composition have currently been studied in various ways.

### CITATION LIST

### Patent Literature

Patent Literature 1: US 2014/0234777 A
Patent Literature 2: US 2013/0012618 A

### SUMMARY OF INVENTION

### Technical Problem

However, in the existing configuration using, for example, an adhesion aid, there is room for improvement in a balance between adhesion to a specific substrate and properties (for example, wiring corrosiveness, storage stability, and solvent solubility) after a highly accelerated stress test (HAST) or other performances with necessary factors such as patterning performance (for example, resolution and alkali developability).

The present invention further improves the balance of various performances of the above-mentioned existing technologies, and an object of the present invention is to provide a photosensitive resin composition capable of forming a resin film excellent in substrate adhesion, particularly adhesion to a Cu-sputtered substrate (Cu adhesion), and having high solvent solubility, to provide a patterned resin film obtained from the photosensitive resin composition and a method for producing the same, and to provide a semiconductor circuit board including a patterned resin film having high HAST resistance or excellent patterning performance.

### Solution to Problem

The present inventors have conducted intensive studies in order to solve the above problems. As a result, the present inventors have found that the above problems can be solved by a photosensitive resin composition containing a compound having a specific nitrogen-containing heterocyclic structure and a crosslinking compound that reacts with the compound by heat, and have completed the present invention. Examples of aspects of the present invention are shown in the following [1] to [9].

[1]

A photosensitive resin composition containing:
(A) an alkali-soluble polymer;
(B) a photosensitizer;
(C) a crosslinking compound having a crosslinkable group that reacts with a component (D) below by heat;
(D) a compound represented by a following general formula (1); and
(E) a solvent:

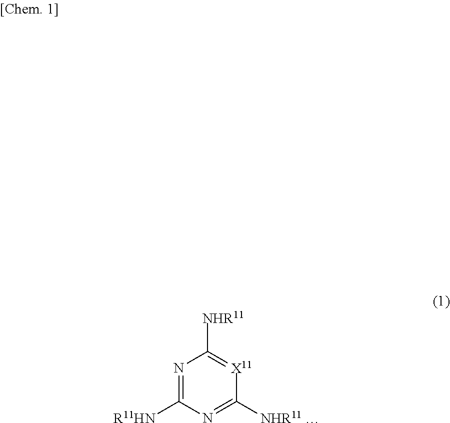

in the general formula (1),
X¹¹ represents CR¹² (R¹² represents a hydrogen atom or a monovalent organic group) or a nitrogen atom,
three R¹¹'s independently represent a hydrogen atom or a group represented by a following general formula (2),

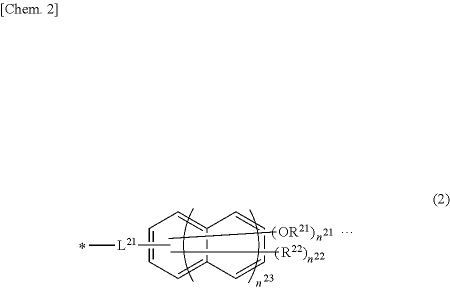

in the general formula (2),
* represents a bond to a nitrogen atom in the general formula (1),
L²¹ represents a single bond or an alkanediyl group having 1 to 5 carbon atoms,
R²¹ represents a hydrogen atom or a hydrocarbon group having 1 to 10 carbon atoms,
R²² represents a monovalent organic group,
n²¹ and n²² each independently represent an integer of 0 or more, and when n²² is an integer of 2 or more, a plurality of R²²'s independently represent the group or represent a ring structure represented by combining a plurality of R²²'s together,
n²³ represents an integer of 0 or 1,
at least one of the three R¹¹'s is a group represented by the general formula (2), and in at least one group represented by the general formula (2), n²¹ is an integer of 1 or more, and at least one R²¹ is a hydrogen atom.
   [2]

The photosensitive resin composition according to [1], in which the photosensitizer (B) is a compound having a quinonediazide group.

[3]

The photosensitive resin composition according to [1] or [2], further containing a silane coupling agent.

[4]

The photosensitive resin composition according to any one of [1] to [3], in which the alkali-soluble polymer (A) is at least one type selected from a group consisting of a polyimide, a polyimide precursor, a polybenzoxazole, a polybenzoxazole precursor, a polymer including a structural unit (d) represented by a following general formula (d), and a novolac resin:

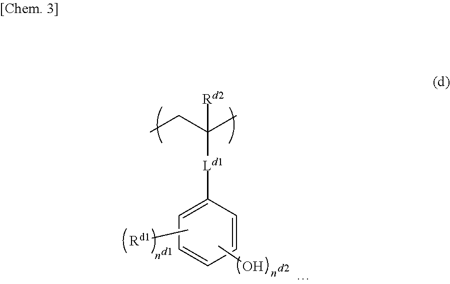

in the general formula (d), L^{d1} represents a single bond, an ester bond, or an amide bond, R^{d1} represents an alkyl group having 1 to 5 carbon atoms or an alkoxy group having 1 to 5 carbon atoms, R^{d2} represents a hydrogen atom or an alkyl group having 1 to 5 carbon atoms, n^{d1} represents an integer of 0 to 4, and n^{d2} represents 1 or 2 (provided that n^{d1}+n^{d2} is an integer of 5 or less).
   [5]

The photosensitive resin composition according to any one of [1] to [4], in which in the general formula (1), X¹¹ is a methine group (CH) or a nitrogen atom.

[6]

The photosensitive resin composition according to any one of [1] to [5], in which in the general formula (1), two or more of the three R¹¹'s are groups represented by the general formula (2).

[7]

A method for producing a resin film having a pattern, the method including: a step (1) of forming, on a substrate, a coating film of the photosensitive resin composition according to any one of [1] to [6]; a step (2) of selectively exposing the coating film; and a step (3) of developing the coating film after exposure with an alkaline developer.

[8]

A resin film having a pattern obtained by curing the photosensitive resin composition according to any one of [1] to [6].

[9]

A semiconductor circuit board including the resin film having a pattern according to [8].

### Advantageous Effects of Invention

According to the present invention, it is possible to provide a photosensitive resin composition capable of forming a resin film excellent in substrate adhesion, particularly adhesion to a Cu-sputtered substrate (Cu adhesion), and having high solubility in a solvent, and further to provide a patterned resin film obtained from the photosensitive resin composition and a method for producing the same, and a semiconductor circuit board including a patterned resin film having high HAST resistance or excellent patterning performance.

### DESCRIPTION OF EMBODIMENTS

Hereinafter, with respect to the present invention, embodiments for carrying out the present invention will be described in detail including preferred embodiments.

### [Photosensitive Resin Composition]

A photosensitive resin composition of the present invention (hereinafter also simply referred to as “composition of the present invention”) contains an alkali-soluble polymer (A), a photosensitizer (B), a crosslinking compound (C) having a crosslinkable group that reacts with a component (D) by heat, a compound (D) represented by the general formula (1), and a solvent (E).

### <Alkali-Soluble Polymer (A)>

The alkali-soluble polymer (A) (hereinafter also simply referred to as “polymer (A)”) contained in the composition of the present invention means that the polymer can be dissolved or swollen in an alkaline developer such as a tetramethylammonium hydroxide aqueous solution having a concentration of 2.38 mass %.

The polymer (A) contained in the composition of the present invention is preferably at least one type of polymer (resin) selected from a group consisting of a polyimide, a polyimide precursor, a polybenzoxazole, a polybenzoxazole precursor, a polymer including a structural unit (d) represented by the general formula (d), and a novolac resin. As the polymer (A), one type may be used, or a plurality of types may be used in combination for the purpose of adjusting the electrical properties such as the relative permittivity of a cured film obtained and the physical properties such as the stress of the film to desired values.

### <Polyimide, Polyimide Precursor>

The polyimide and the polyimide precursor include a structural unit (a) derived from an acid anhydride and a structural unit (b) derived from a diamine. The polyimide and the polyimide precursor may contain an amide bond in the main chain.

### Structural Unit (a)

The structural unit (a) is a structural unit derived from an acid anhydride, and preferably includes at least one type of structural unit derived from an acid anhydride represented by the following formula (3). As the structural unit, one type or two or more types may be included.

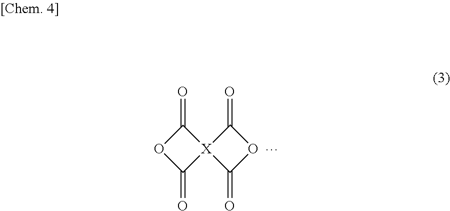

In the formula (3), examples of X include groups represented by the following formulae. In the following formula, * represents a bond to a carbon atom in the formula (3).

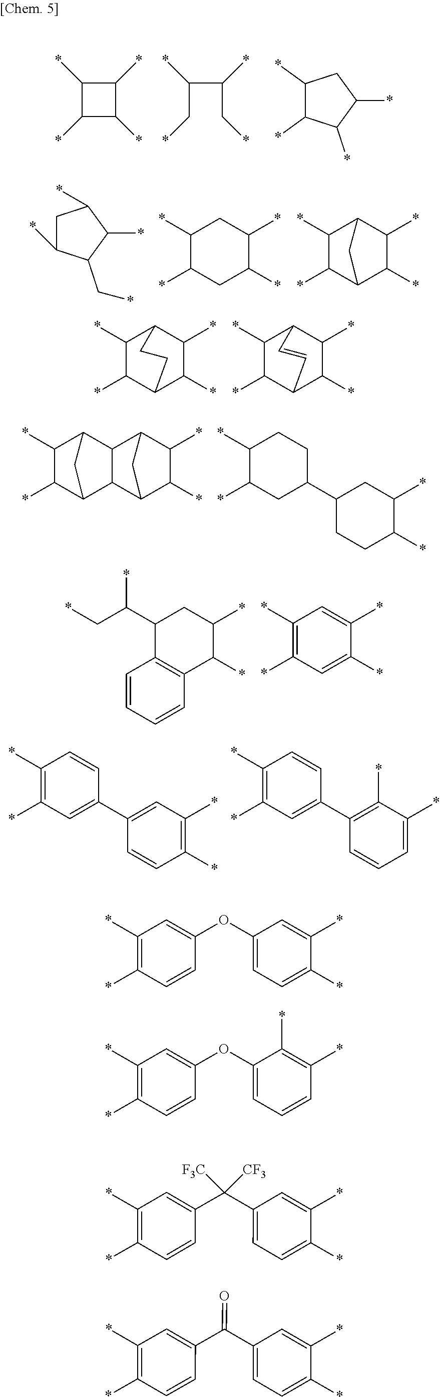

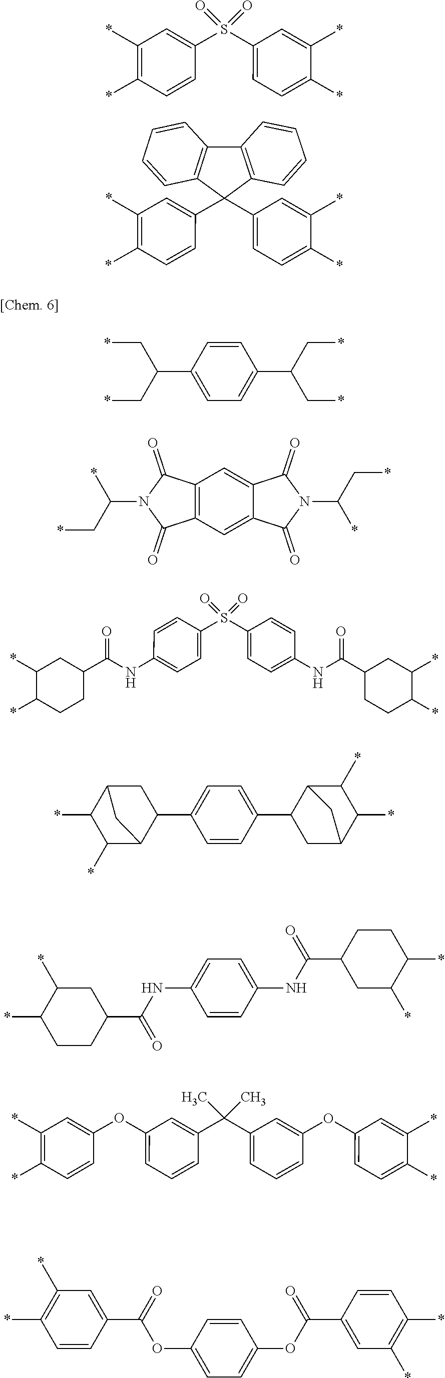

A hydrogen atom (omitted in the formula) in the group represented by the formula may be substituted with, for example, an alkyl group or alkoxy group having 1 to 6 carbon atoms, or a group in which a hydrogen atom in the alkyl group or alkoxy group is substituted with a halogen atom (for example, a trifluoromethyl group).

### Structural Unit (b)

The structural unit (b) is a structural unit derived from a diamine. The structural unit (b) is not particularly limited as long as it is a structural unit derived from a diamine, but examples of the structural unit (b) include a structural unit (b1) derived from a diamine having a phenolic hydroxy group and a structural unit (b2) derived from a diamine having no phenolic hydroxy group, and it is preferable to include a structural unit (b1) derived from a diamine having a phenolic hydroxy group. As the structural unit (b), one type or two or more types may be included.

Examples of the diamine having a phenolic hydroxy group include a diamine represented by the following formula (4).

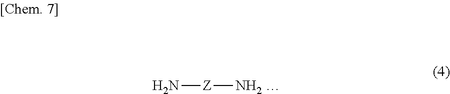

In the formula (4), Z represents a divalent group having a phenolic hydroxy group, and specific examples thereof include divalent groups having a phenolic hydroxy group represented by the following formulae (4-1) to (4-23) In the following formulae (4-1) to (4-23), * represents a bond to a nitrogen atom in the formula (4).

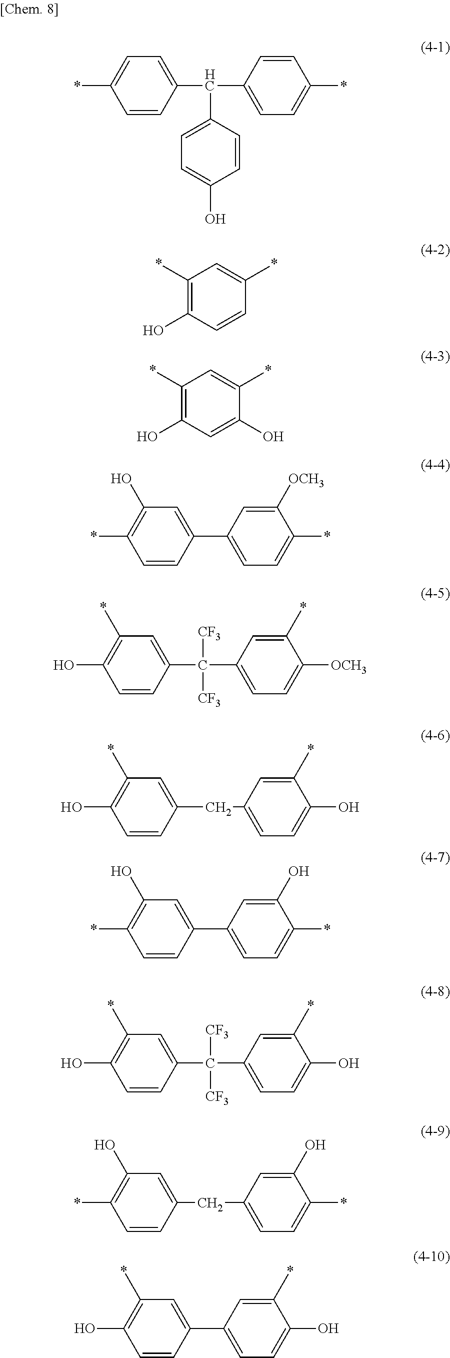

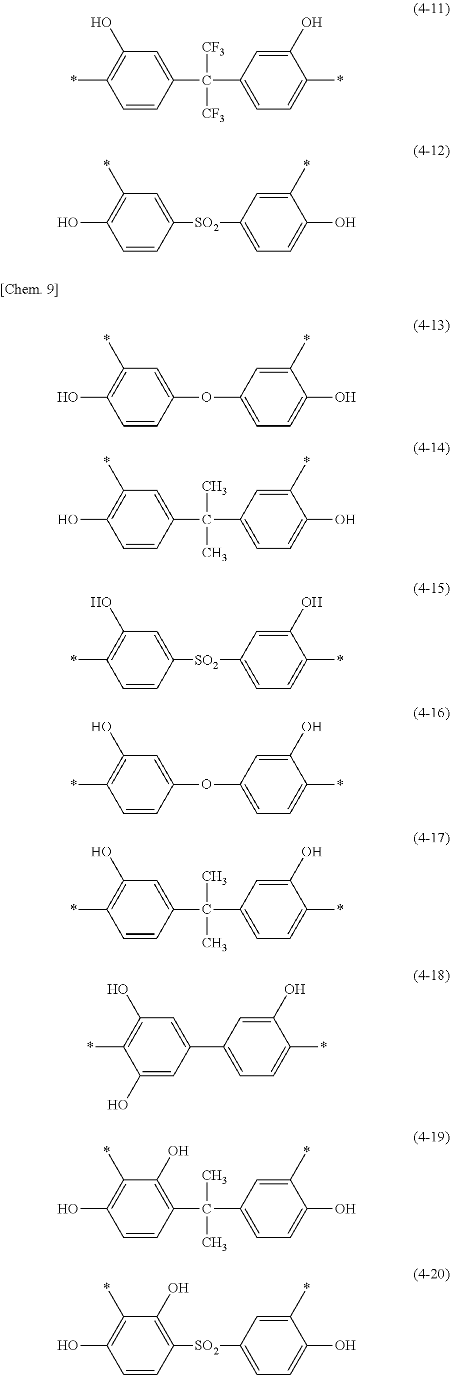

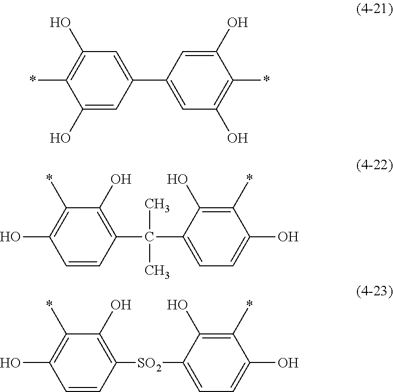

Examples of the diamine that does not have a phenolic hydroxy group and gives the structural unit (b2) derived from a diamine having no phenolic hydroxy group include aromatic diamines such as p-phenylenediamine (1,4-phenylenediamine), m-phenylenediamine (1,3-phenylenediamine), m-tolidine, 2,2′-bis(trifluoromethyl)benzidine, 4,4′-diaminodiphenylmethane, 4,4′-diaminodiphenylethane, 4,4′-diaminodiphenyl sulfide, 4,4′-diaminodiphenyl sulfone, 3,3′-dimethyl-4,4′-diaminobiphenyl, 4,4′-diaminobenzanilide, 4,4′-diaminodiphenyl ether, 1,5-diaminonaphthalene, 2,2′-dimethyl-4,4′-diaminobiphenyl, 5-amino-1-(4′-aminophenyl)-1,3,3-trimethylindane, 6-amino-1-(4′-aminophenyl)-1,3,3-trimethylindane, 3,4′-diaminodiphenyl ether, 3,3′-diaminobenzophenone, 3,4′-diaminobenzophenone, 4,4′-diaminobenzophenone, 1,3-bis(4-aminophenoxy)propane, 1,4-bis(4-aminophenoxy)butane, 1,5-bis(4-aminophenoxy)pentane, 1,6-bis(4-aminophenoxy)hexane, 2,2-bis[4-(4-aminophenoxy)phenyl]propane, 2,2-bis[4-(4-aminophenoxy)phenyl]hexafluoropropane, 2,2-bis(4-aminophenyl)hexafluoropropane, bis[4-(4-aminophenoxy)phenyl]sulfone, 1,4-bis(4-aminophenoxy)benzene, 1,3-bis(4-aminophenoxy)benzene, 1,3-bis(3-aminophenoxy)benzene, 9,9-bis(4-aminophenyl)-10-hydroanthracene, 2,7-diaminofluorene, 9,9-bis(4-aminophenyl)fluorene, 4,4′-methylene-bis(2-chloroaniline), 2,2′,5,5′-tetrachloro-4,4′-diaminobiphenyl, 2,2′-dichloro-4,4′-diamino-5,5′-dimethoxybiphenyl, 3,3′-dimethoxy-4,4′-diaminobiphenyl, 1,4,4′-(p-phenyleneisopropylidene)bisaniline, 4,4′-(m-phenyleneisopropylidene)bisaniline, 2,2′-bis[4-(4-amino-2-trifluoromethylphenoxy)phenyl]hexafluoropropane, 4,4′-bis(4-aminophenoxy)biphenyl, 4,4′-diamino-2,2′-bis(trifluoromethyl)biphenyl, and 4,4′-bis[(4-amino-2-trifluoromethyl)phenoxy]-octafluorobiphenyl; aliphatic and alicyclic diamines such as meta-xylylenediamine, para-xylylenediamine, 1,3-propanediamine, tetramethylenediamine, pentamethylenediamine, hexamethylenediamine, heptamethylenediamine, octamethylenediamine, nonamethylenediamine, decamethylenediamine, dodecamethylenediamine, 4,4′-diaminoheptamethylenediamine, 1,4-diaminocyclohexane, 1,3-bis(aminomethyl)cyclohexane, 1,4-bis(aminomethyl)cyclohexane, isophoronediamine, tetrahydrodicyclopentadienylenediamine, hexahydro-4,7-methanoindanylenedimethylenediamine, tricyclo[6.2.1.02,7]-undecylenedimethyldiamine, and 4,4′-methylenebis(cyclohexylamine); siloxane-containing diamines such as 1,3-bis(3-aminopropyl)tetramethyldisiloxane; polyether diamines; and polyoxyalkylenediamines.

The content molar ratio of the structural unit (a) to the structural unit (b) in the polymer (A) (structural unit (a)/structural unit (b)) is preferably 60/40 to 40/60, and more preferably 55/45 to 45/55. The content ratio of each structural unit can be measured by ¹H-NMR or ¹³C-NMR.

The polymer (A) used in the composition of the present invention preferably has a phenolic hydroxy group in the polymer chain or at the polymer terminal. When a phenolic hydroxy group is introduced into a polymer chain, it is usually achieved by using a monomer having a phenolic hydroxy group as a polymerization component. In addition, when a phenolic hydroxy group is introduced into a polymer terminal, this is usually achieved by using a terminal modifier having a phenolic hydroxy group as a terminal modifier.

Examples of the terminal modifier include 2-aminophenol, 3-aminophenol, and 4-aminophenol. These modify the polymer terminal by a reaction of a reactive terminal (for example, in the case of a polyimide, an acid anhydride, and in the case of a polybenzoxazole described later, a dicarboxylic acid) in the polymer (A) and an amine in the terminal modifier. When a diamine having a phenolic hydroxy group as described above is used as a polymerization monomer, a polymer having a phenolic hydroxy group at the polymer terminal can also be synthesized without using a terminal modifier.

When the terminal modifier is used, the number of moles is preferably set to the number of moles corresponding to the absolute value of the difference between the total number of moles of an acid anhydride group derived from an acid anhydride that gives the structural unit (a) and the total number of moles of an amino group derived from a diamine that gives the structural unit (b) [(number of moles of acid anhydride group)−(number of moles of amino group)].

### <Polybenzoxazole, Polybenzoxazole Precursor>

The polybenzoxazole and the polybenzoxazole precursor include a structural unit (c) derived from a dicarboxylic acid and a structural unit (b1) derived from a diamine having a phenolic hydroxy group. The polybenzoxazole and the polybenzoxazole precursor may contain an amide bond in the main chain.

### Structural Unit (c)

The structural unit (c) is a structural unit derived from a dicarboxylic acid, and as the structural unit (c), one type or two or more types may be included.

Examples of the dicarboxylic acid include diphenyl-4,4′-dicarboxylic acid, terephthalic acid, isophthalic acid, 4,4′-biphenyldicarboxylic acid, benzophenone-4,4′-dicarboxylic acid, 2,2-bis(4-carboxyphenyl)hexafluoropropane, 4,4′-dicarboxydiphenylsulfone, 1,4-cyclohexanedicarboxylic acid, oxalic acid, malonic acid, glutaric acid, suberic acid, and 1,10-decanedicarboxylic acid.

### Structural Unit (b1)

The structural unit (b1) is a structural unit derived from a diamine having a phenolic hydroxy group. As the structural unit (b1), one type or two or more types may be included. Specific examples of the diamine having a phenolic hydroxy group in the polybenzoxazole and the polybenzoxazole precursor include divalent groups having a phenolic hydroxy group represented by formulae (4-2) to (4-23).

It is also possible to add a diamine having no phenolic hydroxy group for the purpose of adjusting, for example, the solubility of the polymer in the solvent or the mechanical properties of a cured film. Specific examples of the diamine having no phenolic hydroxy group include ones similar to those exemplified in the polyimide and the polyimide precursor.

The polybenzoxazole and the polybenzoxazole precursor may have an imide structure or an amic acid or amic acid ester structure in a part of the polymer. Such a polymer is usually achieved by using an acid anhydride that gives the structural unit (a) in combination with a dicarboxylic acid that gives the structural unit (c) and a diamine that has a phenolic hydroxy group and gives the structural unit (b1) during polymerization.

The content molar ratio of the structural unit (c) to the structural unit (b1) in the polymer (A) (structural unit (c)/structural unit (b1)) is preferably 60/40 to 40/60, and more preferably 55/45 to 45/55. The content ratio of each structural unit can be measured by ¹H-NMR or ¹³C-NMR.

As described above, the polymer (A) used in the composition of the present invention preferably has a phenolic hydroxy group in the polymer chain or at the polymer terminal. Also in the polybenzoxazole and the polybenzoxazole precursor, a phenolic hydroxy group can be introduced into the polymer terminal using a terminal modifier having a phenolic hydroxy group as the terminal modifier. Specific examples of the terminal modifier include ones similar to those exemplified in the polyimide and the polyimide precursor.

When the terminal modifier is used, the number of moles is preferably set to the number of moles corresponding to the absolute value of the difference between the total number of moles of a carboxyl group derived from a dicarboxylic acid that gives the structural unit (c) and the total number of moles of an amino group derived from a diamine that has a phenolic hydroxy group and gives the structural unit (b1) [(number of moles of carboxyl group)−(number of moles of amino group)].

<Polymer Including Structural Unit (d)>

### Structural Unit (d)

The structural unit (d) is a structural unit derived from a polymerizable monomer having a phenolic hydroxy group, and is represented by the following general formula (d). As the structural unit (d), one type or two or more types may be included in the polymer.

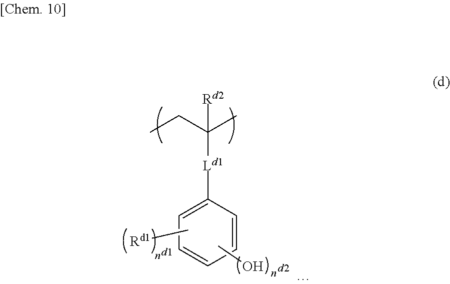

In the general formula (d), L^{d1} represents a single bond, an ester bond, or an amide bond, R^{d1} represents an alkyl group having 1 to 5 carbon atoms or an alkoxy group having 1 to 5 carbon atoms, R^{d2} represents a hydrogen atom or an alkyl group having 1 to 5 carbon atoms, n^{d1} represents an integer of 0 to 4, and n^{d2} represents 1 or 2 (provided that n^{d1}+n^{d2} is an integer of 5 or less).

Examples of the polymer including the structural unit (d) include a homopolymer or a copolymer of a polymerizable monomer that gives the structural unit (d); and a copolymer of a polymerizable monomer that gives the structural unit (d) and another polymerizable monomer. The copolymer may be either a random copolymer or a block copolymer.

Examples of the polymerizable monomer that gives the structural unit (d) include hydroxystyrene-based monomers such as o-hydroxystyrene, m-hydroxystyrene, p-hydroxystyrene, o-isopropenylphenol, m-isopropenylphenol, and p-isopropenylphenol; a monomer obtained by substituting one or two or more hydrogen atoms bonded to aromatic ring carbon of a hydroxystyrene-based monomer with alkyl, alkoxy, halogen, haloalkyl, nitro, or cyano; vinylhydroquinone, 5-vinylpyrogallol, and 4-vinylpyrogallol. Additional examples include a monomer obtained by protecting a phenolic hydroxy group of the monomer with, for example, an alkyl group or a silyl group. When the monomer obtained by protecting a phenolic hydroxy group is used, a polymer having a phenolic hydroxy group can be obtained by removing the protection after a polymer is formed. As the polymer, one type may be used or two or more types may be used in combination.

Examples of another polymerizable monomer include styrene-based monomers such as styrene, α-methylstyrene, o-vinylbenzyl glycidyl ether, m-vinylbenzyl glycidyl ether, and p-vinylbenzyl glycidyl ether; a monomer obtained by substituting one or two or more hydrogen atoms bonded to aromatic ring carbon of a styrene-based monomer with alkyl, alkoxy, halogen, haloalkyl, nitro, or cyano; dienes such as butadiene, isoprene, and chloroprene; unsaturated carboxylic acids such as an unsaturated monocarboxylic acid, an unsaturated dicarboxylic acid, and a half ester thereof; o-vinylbenzoic acid, m-vinylbenzoic acid, p-vinylbenzoic acid, or a monomer obtained by substituting one or two or more hydrogen atoms bonded to aromatic ring carbon of such a monomer with alkyl, alkoxy, halogen, haloalkyl, nitro, or cyano; (meth)acrylic acid esters such as glycidyl (meth)acrylate, (meth)acrylonitrile, acrolein, vinyl chloride, vinylidene chloride, vinyl fluoride, vinylidene fluoride, N-vinylpyrrolidone, vinylpyridine, vinyl acetate, N-phenylmaleimide, N-cyclohexylmaleimide, N-(meth)acryloylphthalimide, and (3,4-epoxycyclohexyl) methyl (meth)acrylate. As the polymer, one type may be used or two or more types may be used in combination.

When the polymer including the structural unit (d) is a copolymer of a polymerizable monomer that gives the structural unit (d) and another polymerizable monomer, the content ratio of the structural unit derived from the polymerizable monomer that gives the structural unit (d) is preferably 30 mol % or more, and more preferably 40 to 95 mol % with respect to 100 mol % of the total content ratio of the structural unit derived from the polymerizable monomer that gives the structural unit (d) and the structural unit derived from another polymerizable monomer.

Preferred examples of the polymer including the structural unit (d) include a hydroxystyrene-based polymer, and specifically include a homopolymer of p-hydroxystyrene; random copolymers such as a m-hydroxystyrene/p-hydroxystyrene copolymer, a p-hydroxystyrene/styrene copolymer, a p-hydroxystyrene/vinylbenzyl glycidyl ether/styrene copolymer, a p-hydroxystyrene/(3,4-epoxycyclohexyl)methyl methacrylate copolymer, and a p-hydroxystyrene/(3,4-epoxycyclohexyl)methyl methacrylate/styrene copolymer; block copolymers such as a p-hydroxystyrene/butadiene block copolymer, a p-hydroxystyrene/isoprene block copolymer, a p-hydroxystyrene/butadiene/p-hydroxystyrene block copolymer, and a p-hydroxystyrene/isoprene/p-hydroxystyrene block copolymer; and hydrogenated block copolymers in which some or all of unsaturated double bonds contained in these block copolymers are hydrogenated.

The block copolymer can be expected to impart, to the resin film obtained from the composition of the present invention, functions such as lowering the relative permittivity of a cured film obtained from the composition of the present invention and relaxing the film stress, in addition to imparting alkali solubility by the phenolic hydroxy group.

### <Novolac Resin>

The novolac resin can be obtained, for example, by condensing a phenol and an aldehyde in the presence of an acid catalyst.

Examples of the phenol include phenol, o-cresol, m-cresol, p-cresol, o-ethylphenol, m-ethylphenol, p-ethylphenol, o-butylphenol, m-butylphenol, p-butylphenol, 2,3-xylenol, 2,4-xylenol, 2,5-xylenol, 2,6-xylenol, 3,4-xylenol, 3,5-xylenol, 2,3,5-trimethylphenol, 3,4,5-trimethylphenol, catechol, resorcinol, pyrogallol, α-naphthol, and β-naphthol.

Examples of the aldehyde include formaldehyde, paraformaldehyde, acetaldehyde, benzaldehyde, and salicylaldehyde.

Specific examples of the novolac resin include a phenol/formaldehyde condensed novolac resin, a cresol/formaldehyde condensed novolac resin, a cresol/salicylaldehyde condensed novolac resin, a phenol-naphthol/formaldehyde condensed novolac resin, and a resin obtained by modifying a novolac resin with a rubber-like polymer having a polymerizable vinyl group such as a butadiene-based polymer (for example, a resin described in JP 2010-015101 A).

### (Properties of Polymer (A))

The polymer (A) usually has a polystyrene equivalent weight average molecular weight (Mw) as measured by gel permeation chromatography (GPC) of about 2,000 to 800,000. When the polymer (A) is used in the photosensitive resin composition, the Mw is preferably about 2,000 to 100,000, more preferably about 2,000 to 70,000, and still more preferably about 3,000 to 50,000. When the Mw is less than 2,000, there is a tendency that sufficient mechanical properties as an insulation film cannot be obtained. Meanwhile, when the Mw is more than 100,000, the solubility of the exposed portion of the photosensitive resin composition obtained using the polymer (A) in a solvent or a developer tends to be poor.

As the polymer (A), one type can be used or two or more types can be used in combination.

A lower limit of a content ratio of the polymer (A) in 100 mass % of a solid content of the composition of the present invention is preferably 20 mass %, more preferably 40 mass %, and still more preferably 60 mass %, and an upper limit thereof is preferably 99 mass % and more preferably 95 mass %. Note that the solid content refers to all components other than a solvent (E) described below that can be contained in the composition of the present invention.

### <Method for Producing Polymer (A)>

The polymer (A) each can be produced by a known method.

For example, in the case of a polyimide and a polyimide precursor, according to a known method, a polyimide can be obtained by causing a reaction in a polymerization solvent to synthesize a polyamic acid using an acid anhydride and a diamine, and further causing an imidization reaction (for example, a heat imidization reaction or a chemical imidization reaction) to synthesize a polyimide. At this time, the polyamic acid synthesized during the process can be the polymer (A) having the polyimide precursor structure described above. In addition, a polyamic acid ester obtained by esterifying the polyamic acid according to a known method can also be the polymer (A) having the polyimide precursor structure described above.

In addition, for example, in the case of a polybenzoxazole and a polybenzoxazole precursor, according to a known method, a polybenzoxazole can be synthesized by causing a reaction of a dicarboxylic acid (or a dicarboxylic acid chloride or a dicarboxylic acid active ester corresponding to the dicarboxylic acid) and a diamine having a phenolic hydroxy group to synthesize a polyhydroxyamide, and further causing a dehydration ring-closing reaction (for example, a heat treatment or a chemical treatment). At this time, the polyhydroxyamide synthesized during the process can be the polymer (A) having the polybenzoxazole precursor structure described above.

The polymer including the structural unit (d) other than the above polymers and the novolac resin each can also be produced by a known polymerization reaction.

The structure included in the polymer (A) produced can be checked by, for example, 1H-NMR or ¹³C-NMR.

### <Photosensitizer (B)>

The composition of the present invention contains a photosensitizer (B). The photosensitizer (B) is a compound that functions as a photoacid generator and generates an acid by a treatment including light irradiation. By the exposure treatment of the resin film formed from the composition of the present invention, an acid is generated in the exposed portion based on the photosensitizer (B), and the solubility of the exposed portion in an alkaline developer changes based on the action of the acid.

The resin film obtained from the composition containing the photosensitizer (B) is a film poorly soluble in an alkaline developer. On the other hand, the photosensitizer (B) generates a carboxylic acid by a treatment including light irradiation and development using an alkaline developer. Therefore, a positive pattern can be formed using the photosensitizer (B).

The photosensitizer (B) is preferably a compound having a quinonediazide group, and examples thereof include an ester compound of a compound having one or more phenolic hydroxy groups with 1,2-naphthoquinonediazide-4-sulfonic acid or 1,2-naphthoquinonediazide-5-sulfonic acid. Specific examples of the compound having one or more phenolic hydroxy groups include compounds described in paragraphs [0065] to [0070] of JP 2014-186300 A, and these compounds shall be described herein.

As the photosensitizer (B), one type can be used or two or more types can be used in combination.

In the composition of the present invention, the content of the photosensitizer (B) is usually 5 to 50 parts by mass, preferably 10 to 40 parts by mass, and more preferably 15 to 35 parts by mass with respect to 100 parts by mass of the polymer (A). When the content of the photosensitizer (B) is the above lower limit value or more, the residual film rate of the unexposed portion is improved, and an image that is faithful to a pattern mask is easily obtained. When the content of the photosensitizer (B) is the above upper limit value or less, a resin film having an excellent pattern shape is easily obtained, and there is a tendency that foaming during film formation can be prevented.

<Crosslinking Compound (C) Having Crosslinkable Group Reacting with Component (D) by Heat>

The composition of the present invention contains a crosslinking compound (C) having a crosslinkable group that reacts with a component (D) described later by heat (hereinafter also simply referred to as “crosslinking compound (C)”). The crosslinking compound (C) is used as a crosslinking agent for improving the HAST resistance of a cured film, improving the curability of a resin film, and forming an excellent pattern shape. The crosslinking agent acts also as a crosslinking component (curing component) that reacts also with the polymer (A) or reacts between the crosslinking agents. The crosslinking compound (C) has a crosslinkable group that reacts with the component (D) by heat, and therefore reacts also with the component (D) in addition to the action. Examples of the crosslinkable group that reacts with the component (D) by heat include a methylol group, an alkoxymethyl group, an oxetane ring, an oxirane ring, an oxazoline ring, a cyanate ester group, and a maleimide group.

Examples of such a crosslinking compound (C) include a crosslinking compound having at least two methylol groups or alkoxymethyl groups, a crosslinking compound having at least two oxetane rings, a crosslinking compound having at least two oxirane rings, a crosslinking compound having at least two oxazoline rings, a crosslinking compound having at least two cyanate ester groups, a crosslinking compound having at least two maleimide groups, and a compound having at least two different crosslinkable groups in total among the above-described crosslinkable groups. Among them, a crosslinking compound having at least two methylol groups or alkoxymethyl groups, a crosslinking compound having at least two oxirane rings, a crosslinking compound having at least two cyanate ester groups, and a crosslinking compound having at least two maleimide groups are preferable.

For example, in the crosslinking compound having at least two methylol groups or alkoxymethyl groups, the crosslinkable group is formed only of a methylol group or an alkoxymethyl group, and a crosslinking compound containing other crosslinkable groups (for example, an oxetane ring, an oxirane ring, an oxazoline ring, a cyanate ester group, and a maleimide group) is not included. The same applies to a crosslinking compound having at least two oxetane rings, a crosslinking compound having at least two oxirane rings, a crosslinking compound having at least two oxazoline rings, a crosslinking compound having at least two cyanate ester groups, and a crosslinking compound having at least two maleimide groups.

The methylol group or alkoxymethyl group is a group represented by —CH₂OR.

In the formula, R is a hydrogen atom, an alkyl group having 1 to 10 carbon atoms, or an acyl group having 2 to 10 carbon atoms, preferably a hydrogen atom or an alkyl group having 1 to 6 carbon atoms. Examples of the alkyl group include a methyl group, an ethyl group, a propyl group, and a butyl group. Examples of the acyl group include an acetyl group. Here, it is considered that in the group represented by —CH₂OR, a methylene group reacts with the phenolic hydroxy group or its ortho position of the polymer (A) or the component (D), so that the crosslinking reaction proceeds.

Examples of the crosslinking compound having at least two methylol groups or alkoxymethyl groups include a methylol group-containing phenol compound, an alkylmethylol group-containing phenol compound, and an acyloxymethyl group-containing phenol compound, and specifically, compounds shown below are preferable.

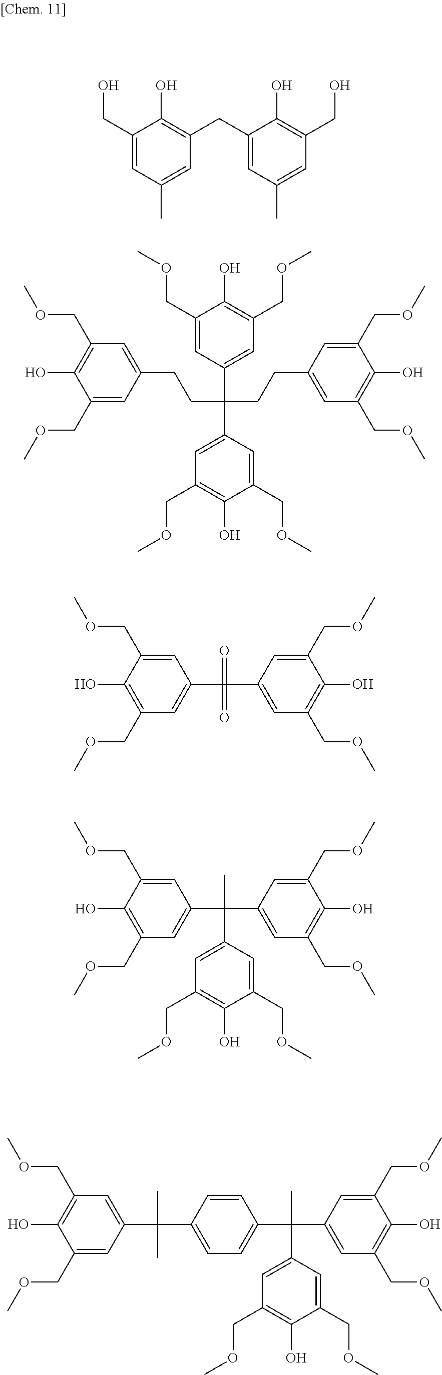

Examples of the crosslinking compound having at least two methylol groups or alkoxymethyl groups other than the above compounds include nitrogen compounds such as polymethylolated melamine, polymethylolated glycoluril, polymethylolated guanamine, and polymethylolated urea; and compounds in which all or some of active methylol groups (CH₂OH groups bonded to nitrogen atoms) in the nitrogen compounds are alkyl-etherified or acyloxylated. Here, examples of the alkyl group constituting the alkyl ether include a methyl group, an ethyl group, a propyl group, and a butyl group, and these groups may be identical to or different from each other. Further, the active methylol group that is not alkyl-etherified or acyloxylated can self-condense within one molecule or can condense between two molecules, resulting in the formation of an oligomer component.

Examples of such a crosslinking compound include crosslinking agents described in JP H06-180501 A, JP 2006-178059 A, and JP 2012-226297 A. Specific examples thereof include melamine-based crosslinking agents such as polymethylolated melamine, hexamethoxymethylmelamine, hexaethoxymethylmelamine, hexapropoxymethylmelamine, and hexabutoxymethylmelamine; glycoluril-based crosslinking agents such as polymethylolated glycoluril, tetramethoxymethylglycoluril, and tetrabutoxymethylglycoluril; guanamine-based crosslinking agents such as compounds, in which guanamine is methylolated, such as 3,9-bis[2-(3,5-diamino-2,4,6-triazaphenyl)ethyl]-2,4,8,10-tetraoxospiro[5.5]undecane and 3,9-bis[2-(3,5-diamino-2,4,6-triazaphenyl)propyl]-2,4,8,10-tetraoxospiro[5.5]undecane, and compounds, in which all or some of active methylol groups in the compounds are alkyl-etherified or acyloxylated; and urea-based crosslinking agents, and specifically, the compounds shown below are preferable.

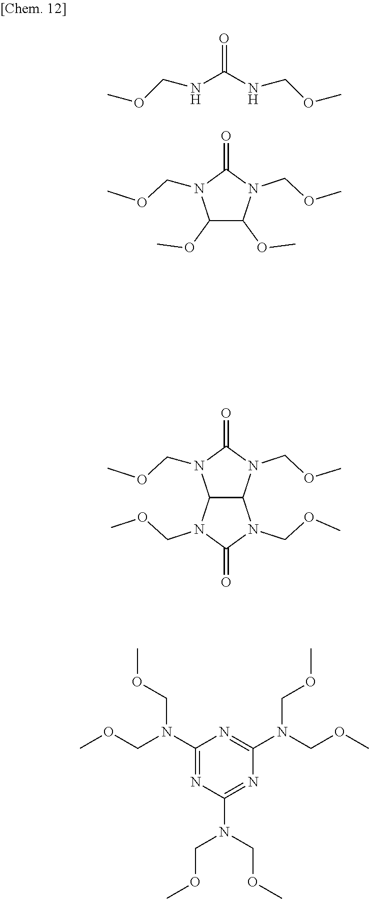

Examples of the crosslinking compound having at least two oxirane rings include ethylene glycol diglycidyl ether, diethylene glycol diglycidyl ether, propylene glycol diglycidyl ether, tripropylene glycol diglycidyl ether, dipentaerythritol hexaglycidyl ether, pentaerythritol tetraglycidyl ether, trimethylolethane triglycidyl ether, trimethylolpropane triglycidyl ether, glycerin triglycidyl ether, 1,4-cyclohexanedimethanol diglycidyl ether, and diglycidyl 1,2-cyclohexanedicarboxylate.

As the crosslinking compound having at least two oxirane rings, a commercially available product may be used. Examples of the commercially available product of the crosslinking agent having at least two oxirane rings include “Epolite 40E”, “Epolite 100E”, “Epolite 70P”, “Epolite 200P”, “Epolite 1500NP”, “Epolite 1600”, “Epolite 80MF”, and “Epolite 100MF” manufactured by Kyoeisha Chemical Co., Ltd.; alkyl epoxy resin “ZX-1542” manufactured by Nippon Steel & Sumikin Chemical Co., Ltd.; “DENACOL (registered trademark) EX-212L”, “DENACOL (registered trademark) EX-214L”, “DENACOL (registered trademark) EX-216L”, “DENACOL (registered trademark) EX-321L”, and “DENACOL (registered trademark) EX-850L” manufactured by Nagase Chemtech Corporation.

Examples of the crosslinking compound having at least two cyanate ester groups include compounds represented by the following formulae (b-6-1) to (b-6-7). In the following formulae (b-6-6) and (b-6-7), n is independently 0 to 30.

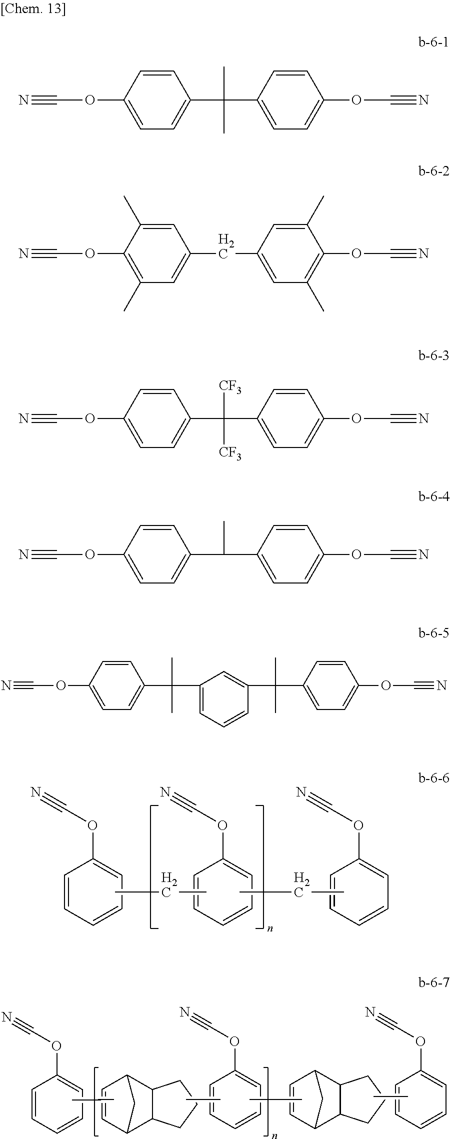

As the crosslinking compound having at least two maleimide groups, a commercially available product may be used. Examples of the commercially available product of the crosslinking agent having at least two maleimide groups include “BMI-2000”, “BMI-2300”, “BMI-TMH”, “BMI-1000”, “BMI-3000”, “BMI-4000”, “BMI-5100”, and “BMI-7000” manufactured by Daiwa Kasei Industry Co., Ltd.; “MIR-3000” and “MIR-5000” manufactured by Nippon Kayaku Co., Ltd., and “SLK-3000”, “SLK-6895”, “SLK-1500”, “SLK-2500”, and “SLK-6100” manufactured by Shin-Etsu Chemical Co., Ltd.

As the crosslinking compound (C), one type can be used or two or more types can be used in combination.

In the composition of the present invention, the content of the crosslinking compound (C) is usually 1 to 60 parts by mass, preferably 3 to 50 parts by mass, and more preferably 5 to 40 parts by mass with respect to 100 parts by mass of the polymer (A). When the content of the crosslinking compound (C) is within the above range, a resin film excellent in pattern shape and heat resistance is formed. In addition, the semiconductor circuit board obtained from the resin film is excellent in HAST resistance.

### <Compound (D)>

The composition of the present invention contains a compound (D) represented by the following general formula (1). Use of the compound (D) can provide a substrate excellent in not only adhesion to a substrate (particularly adhesion to a Cu-sputtered substrate) but also HAST resistance and patterning performance. As the compound (D), one type can be used or two or more types can be used in combination.

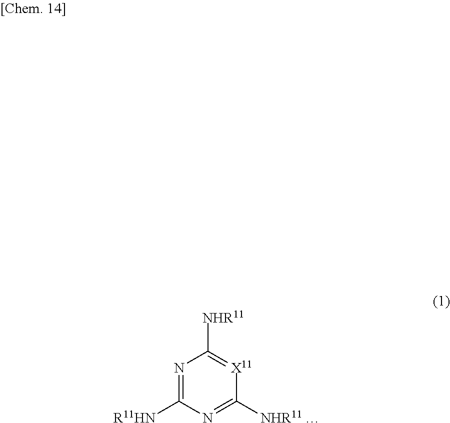

In the general formula (1), X¹¹ represents CR¹² (R¹² represents a hydrogen atom or a monovalent organic group) or a nitrogen atom, and is preferably a methine group (CH) or a nitrogen atom. Examples of the monovalent organic group include a methyl group and an ethyl group.

In the general formula (1), three R¹¹'s independently represent a hydrogen atom or a group represented by the following general formula (2), at least one of the three R¹¹'s is a group represented by the general formula (2), and preferably two or more of the three R¹¹'s are groups represented by the following general formula (2).

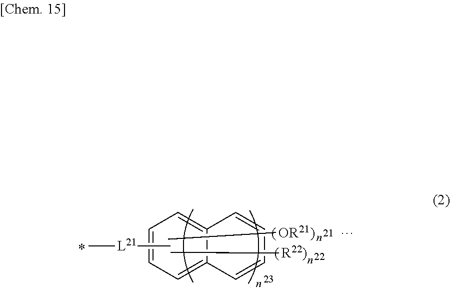

In the general formula (2), * represents a bond to a nitrogen atom in the general formula (1).

In the general formula (2), L²¹ represents a single bond or an alkanediyl group having 1 to 5 carbon atoms, and is preferably a single bond or an ethanediyl group (—CH₂—CH₂—). R²¹ represents a hydrogen atom or a hydrocarbon group having 1 to 10 carbon atoms such as a methyl group or an ethyl group, and is preferably a hydrogen atom. R²² represents a monovalent organic group. Examples of the monovalent organic group include hydrocarbon groups having 1 to 10 carbon atoms such as a methyl group and an ethyl group, an amino group, and a carboxyl group, and hydrocarbon groups having 1 to 10 carbon atoms such as a methyl group and an ethyl group are preferable.

In the general formula (2), n²¹ and n²² each independently represent an integer of 0 or more, and n²³ represents an integer of 0 or 1. As described above, at least one of the three R¹¹'s in the general formula (1) is a group represented by the general formula (2), and in at least one group represented by the general formula (2), n²¹ is an integer of 1 or more, and at least one R²¹ is a hydrogen atom. When n²² is an integer of 2 or more, a plurality of R²²'s independently represent the group or represent a ring structure represented by combining together. When the ring structure is represented, the ring structure is preferably a ring structure represented by combining adjacent R²²'s together.

Specific examples of the compound (D) include compounds shown below.

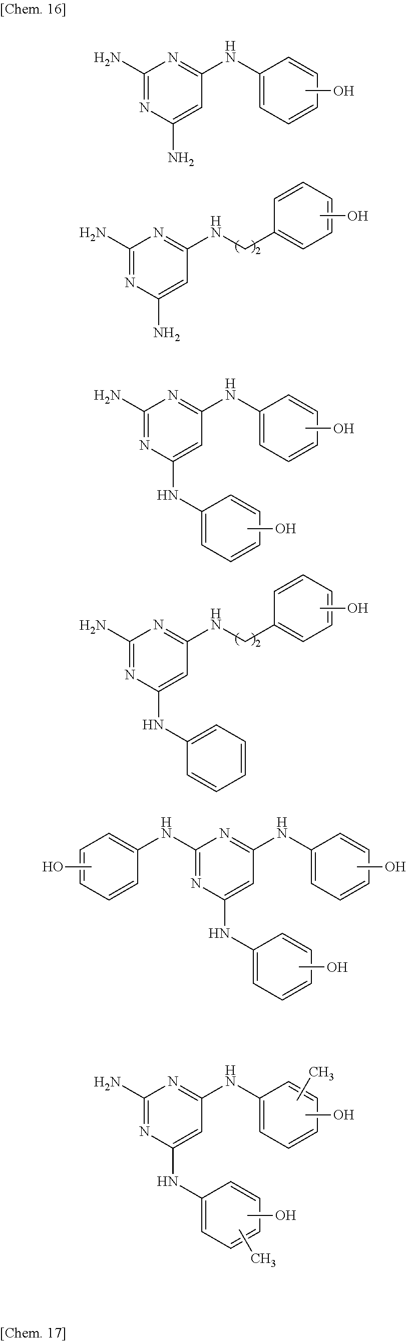

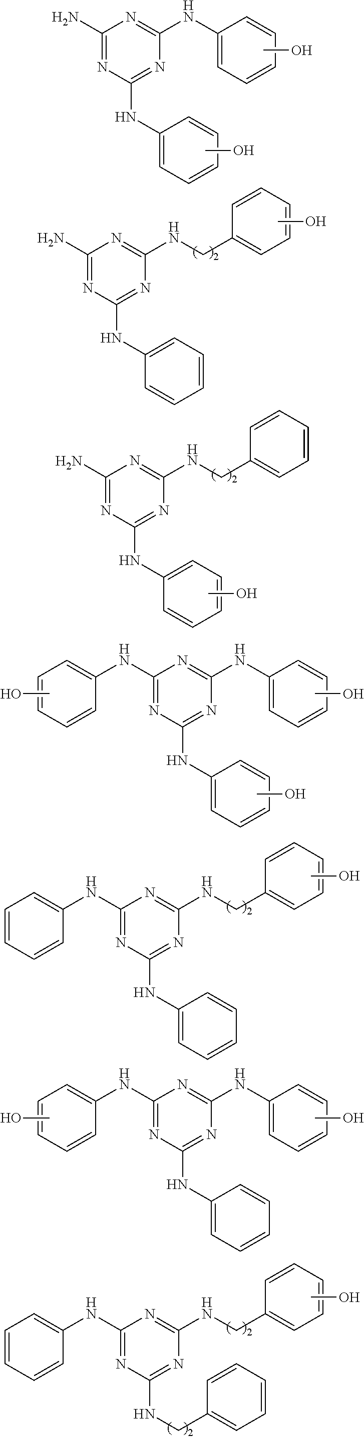

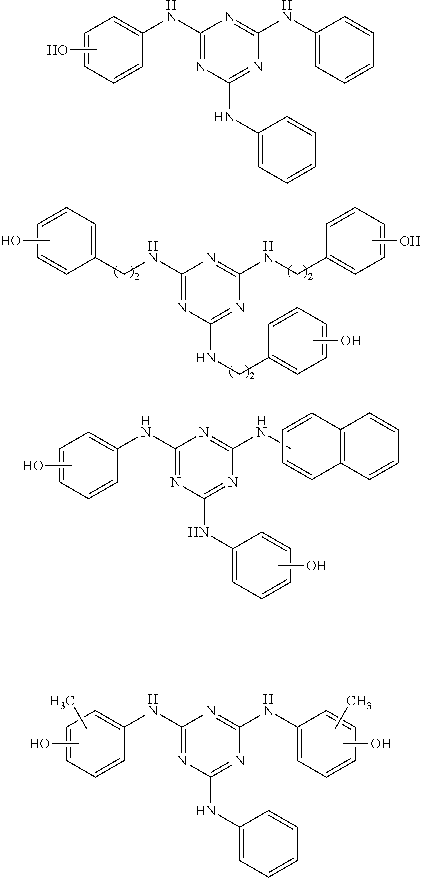

As the compound (D), a compound produced and purified by a known method may be used, or a commercially available product may be used. As the compound (D), compounds shown below are particularly preferable from the viewpoint of availability or ease of production.

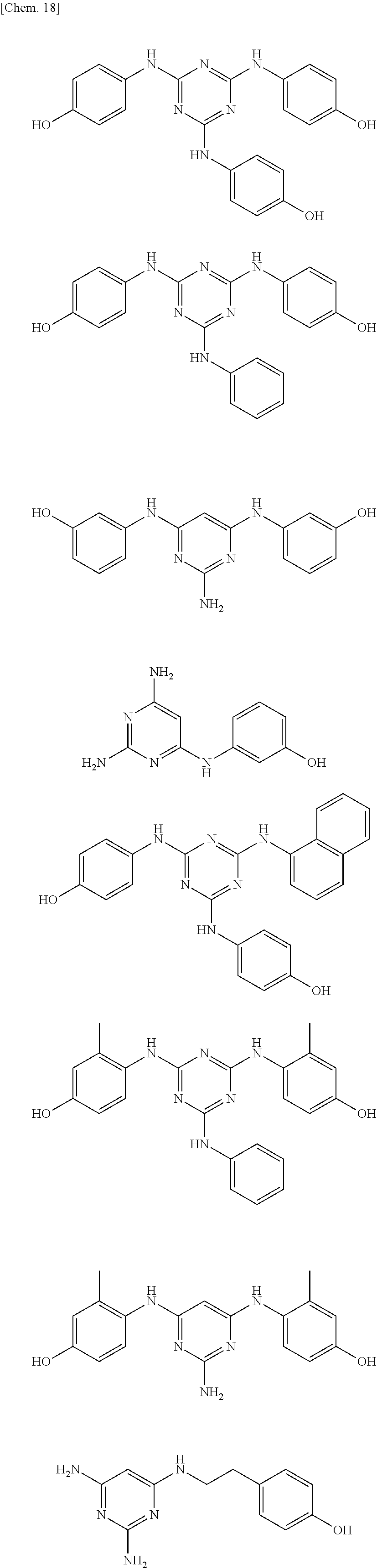

The molecular weight of the compound (D) is preferably 1,000 or less, and more preferably 500 or less. When the molecular weight of the compound (D) is in the above range, the solvent solubility tends to be excellent, so that a uniformly dissolved composition is obtained, and a desired effect is easily exhibited in a cured film.

In the composition of the present invention, the content of the compound (D) is 1 to 40 parts by mass, preferably 1 to 30 parts by mass, and more preferably 2 to 15 parts by mass, with respect to 100 parts by mass of the polymer (A). When the content of the compound (D) is in the above range, a resin film excellent in adhesion to a substrate is formed, and a semiconductor circuit board excellent in HAST resistance and patterning performance tends to be obtained.

One of the reasons why the incorporation of the compound (D) in the composition of the present invention not only provides a resin film excellent in adhesion to a substrate, but also provides a semiconductor circuit board excellent in HAST resistance can be presumed to be that the compound (D) reacts with and binds to the polymer (A) or the crosslinking compound (C), so that, for example, metal ions (example: Cu²⁺) generated at the time of use as a semiconductor circuit board are supplemented and ion transfer (migration) in the film can be prevented.

### <Solvent (E)>

The composition of the present invention contains a solvent (E). Use of the solvent (E) can improve the handleability of the composition of the present invention, and adjust the viscosity and storage stability.

The solvent (E) is not particularly limited as long as it is an organic solvent capable of dissolving or dispersing each component such as the polymer (A), the compound (B), the crosslinking compound (C), the compound (D), and any other components described later. Examples of the solvent (E) include a ketone solvent, an alcohol solvent, an ether solvent, an ester solvent, an amide solvent, a hydrocarbon solvent, and a lactone solvent. The solvent (E) preferably contains at least one type selected from an ester solvent, an amide solvent, and a lactone solvent because of excellent solubility.

Specific examples of the solvent include ester solvents such as ethyl lactate (EL), cyclic amide solvents such as N-methyl-2-pyrrolidone (NMP) and dimethylimidazolidinone (DMI), and cyclic lactone solvents such as γ-butyrolactone (GBL).

In the composition of the present invention, one type or two or more types can be contained as the solvent (E). The content of the solvent (E) in the composition of the present invention is an amount at which a solid content concentration in the composition is usually 10 to 50 mass %.

### <Other Components>

The composition of the present invention may contain other components in addition to the components described above as long as the objects and characteristics of the present invention are not impaired. Examples of the other components include a polymer other than the polymer (A), a crosslinking compound (crosslinking agent) other than the crosslinking compound (C), and additives such as a low-molecular-weight phenol compound, a silane coupling agent, a rust inhibitor, an adhesion aid, crosslinked fine particles, a leveling agent, a sensitizer, an inorganic filler, and a quencher.

### Silane Coupling Agent

The composition of the present invention preferably further contains a silane coupling agent, and more preferably contains at least one silane coupling agent of the following (1) to (3).
(1) A silane coupling agent having an isocyanate group (d1)
(2) A silane coupling agent having an amino group (d2)
(3) A combination of a silane coupling agent represented by the following general formula (d3) and at least one type of silane compound selected from the group consisting of silane compounds represented by the following general formulae (d4) to (d8)

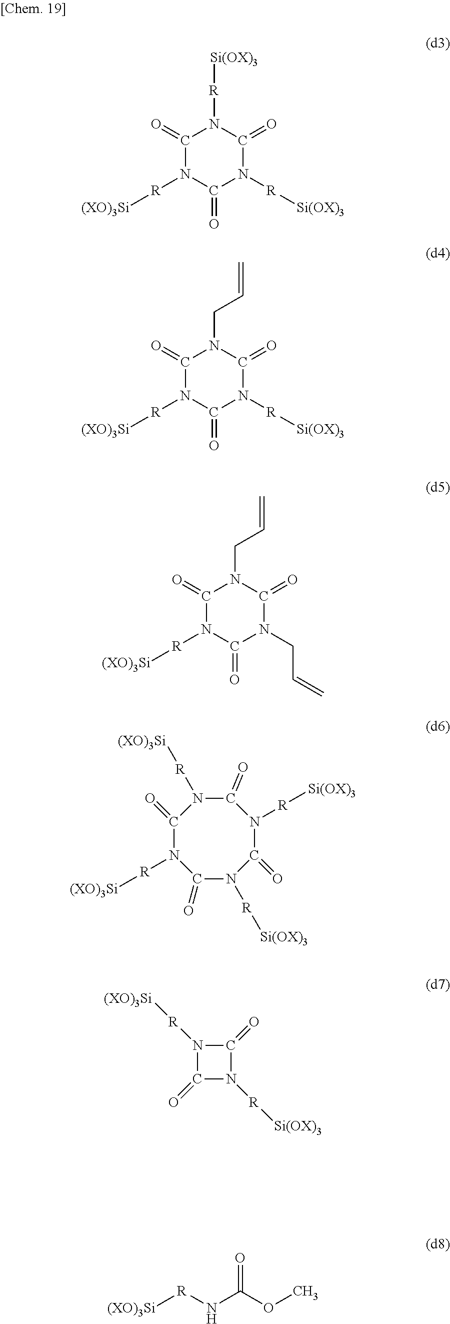

By the incorporation of such a silane coupling agent, the composition of the present invention forms a resin film excellent in adhesion to a substrate and excellent in dry etching resistance.

(Silane Coupling Agent Having Isocyanate Group (d1))

A silane coupling agent having an isocyanate group is conventionally known, and examples thereof include 3-isocyanatopropyltrimethoxysilane and 3-isocyanatopropyltriethoxysilane.

(Silane Coupling Agent Having Amino Group (d2))

A silane coupling agent having an amino group is conventionally known, and is, for example, a compound represented by the following general formula.

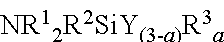

In the formula,
R¹ is a hydrogen atom, a hydrocarbon group having 1 to 4 carbon atoms, or an aryl group having 6 to 10 carbon atoms,
R² is a divalent hydrocarbon group having 2 to 11 carbon atoms, preferably 3 to 5 carbon atoms,
R³ is an alkyl group having 1 to 8 carbon atoms or a halogenated alkyl group, for example, a methyl group, an ethyl group, a propyl group, an isopropyl group, a butyl group, a hexyl group, or a cyclopropyl group; an aryl group having at least 6 ring carbon atoms, for example, a phenyl group, a naphthyl group, or a tolyl group; or an aralkyl group, for example a benzyl group or a phenylethyl group,
a is an integer of 0 to 3,
Y is a group selected from the group consisting of an alkoxy group (R⁴O—); a trialkylsilyloxy group (—OSiR⁴³); and
an alkoxy-substituted alkoxy group (—OR⁵—(OR⁴)ₘ (R⁵ is a divalent hydrocarbon group having 1 to 8 carbon atoms, and m is an integer of 1 to 3)) R⁴'s are independently a substituted or unsubstituted monovalent hydrocarbon group having 1 to 8 carbon atoms, and may be either a straight chain or a branched chain.

Specific examples of such a compound include 3-aminopropyltrimethoxysilane, 3-aminopropyltriethoxysilane, 3-aminopropylmethyldiethoxysilane, 3-aminopropylmethyldimethoxysilane, 3-aminopropylethyldiethoxysilane, 3-aminopropylphenyldiethoxysilane, 3-aminopropylphenyldimethoxysilane, 4-aminobutyltrimethoxysilane, 4-aminobutyltriethoxysilane, 4-aminobutylmethyldiethoxysilane, 4-aminobutylmethyldimethoxysilane, 4-aminobutylethyldiethoxysilane, 4-aminobutylethyldimethoxysilane, 4-aminobutylphenyldiethoxysilane, 4-aminobutylphenyldimethoxysilane, 2-aminoisopropyltrimethoxysilane, 3-aminopropyltripropoxysilane, 3-aminopropyltributoxysilane, 3-aminopropylphenylmethyl-n-propoxysilane, 3-aminopropylmethyldibutoxysilane, 3-aminopropyl-tris(methoxyethoxyethoxy)silane, 3-aminopropyldimethylethoxysilane, 3-aminopropyldiethylmethylsilane, 3-aminopropyl-tris(trimethylsiloxy)silane, ω-aminoundecyltrimethoxysilane, 4-aminobutyldimethylmethoxysilane, 4-amino(3-methylbutyl)methyldimethoxysilane, 4-amino(3-methylbutyl)methyldiethoxysilane, 4-amino(3-methylbutyl)trimethoxysilane, and 3-(N-phenyl)aminopropyltrimethoxysilane.

Among them, 3-aminopropyltrimethoxysilane, 3-(N-phenyl)aminopropyltrimethoxysilane, and 3-aminopropyltriethoxysilane are preferable from the viewpoint of forming a resin film excellent in adhesion to a substrate.

(3) A combination of a silane coupling agent represented by a general formula (d3) and at least one type of silane compound selected from the group consisting of silane compounds represented by general formulae (d4) to (d8) In the silane coupling agent represented by the general formula (d3), R represents a divalent hydrocarbon group, X represents an alkyl group having 1 to 5 carbon atoms, and a plurality of R's and X's may be identical or different.

Examples of the divalent hydrocarbon group include a methylene group and an alkylene group having 2 to 10 carbon atoms. Examples of the alkylene group include an ethylene group, a propylene group, an iso-propylene group, a butylene group, and an iso-butylene group.

Examples of the alkyl group having 1 to 5 carbon atoms include a methyl group, an ethyl group, an isopropyl group, a butyl group, and an iso-butyl group.

Such a compound represented by the general formula (d3) is conventionally known, and examples thereof include tris(3-trimethoxysilylpropyl)isocyanurate, tris(3-triethoxysilylpropyl)isocyanurate, tris(3-tri-i-propoxysilylpropyl)isocyanurate, and tris(3-tributoxysilylpropyl)isocyanurate.

In addition, methods for producing these compounds are also conventionally known, and these compounds can be produced by the production methods described in, for example, U.S. Pat. No. 3,517,001, JP S63-141987 A, and JP H09-195226 A. A commercially available silane coupling agent is also produced by a conventionally known production method, and contains at least one type of the silane compounds represented by the general formulae (d4) to (d8) as an impurity.

In the composition of the present invention, the content of the silane coupling agent is 1 to 10 parts by mass, preferably 1 to 5 parts by mass with respect to 100 parts by mass of the polymer (A). When the content of the silane coupling agent is within the above range, adhesion to the Si substrate tends to be particularly excellent.

### <Method for Producing Photosensitive Resin Composition>

The composition of the present invention can be prepared by uniformly mixing the respective components constituting the composition of the present invention. In addition, in order to remove foreign matter, after uniformly mixing the respective components, the obtained mixture can be filtered with, for example, a filter.

### <Properties of Photosensitive Resin Composition>

A resin film (cured film) obtained by curing the composition of the present invention is excellent in adhesion to a substrate, HAST resistance, and patterning performance. When an aqueous solution containing an alkaline compound is used as the developer, it is preferable to introduce a hydrophilic highly polar functional group such as a phenolic hydroxy group into the repeating structural unit of the polymer or the polymer terminal in the polymer (A) to be used from the viewpoint that it becomes easy to adjust the solubility in the developer as appropriate.

### [Resin Film Having Pattern]

A resin film having a pattern (patterned resin film) of the present invention is obtained by curing the composition of the present invention described above. Specifically, the patterned resin film obtained by the production method described later can be preferably used as an insulation film (examples: a surface protective film, an interlayer insulation film, or a planarization film) included in a semiconductor circuit board.

### <Method for Producing Resin Film Having Pattern>

The resin film having a pattern (patterned resin film) of the present invention can be produced by a method including a step (1) of forming a coating film of the composition of the present invention on a substrate, a step (2) of selectively exposing the coating film, and a step (3) of developing the exposed coating film with an alkaline developer.

### <Step (1)>

In the step (1), the composition of the present invention is usually applied onto a substrate so that a thickness of a finally obtained patterned resin film is, for example, 0.1 to 100 μm. The substrate after application of the composition is usually heated at 50 to 140° C. for 10 to 360 seconds using an oven or a hot plate. As such, a coating film formed using the composition of the present invention is formed on a substrate.

Examples of the substrate include a silicon wafer, a compound semiconductor wafer, a wafer with a metal thin film (for example, a Cu-sputtered Si substrate), a glass substrate, a quartz substrate, a ceramic substrate, an aluminum substrate, and a substrate having a semiconductor chip on a surface of each of these substrates. Examples of the application method include a dipping method, a spraying method, a bar coating method, a roll coating method, a spin coating method, a curtain coating method, a gravure printing method, a silk screen method, and an inkjet method.

### <Step (2)>

In the step (2), the coating film is selectively exposed using, for example, a contact aligner, a stepper, or a scanner. The expression “selectively” specifically means via a photomask on which a predetermined mask pattern is formed.

Examples of the exposure light include ultraviolet rays and visible rays, and light having a wavelength of 200 to 500 nm (for example, i-line (365 nm)) is usually used. An exposure dose by exposure light varies depending on, for example, the type and blending ratio of each component in the composition of the present invention and a thickness of the coating film, and the exposure dose is usually 100 to 1,500 mJ/cm².

### <Step (3)>

In the step (3), the resin film is developed with an alkaline developer, and the exposed portion is dissolved and removed to form a desired patterned on the substrate. Examples of the development method include a shower development method, a spray development method, an immersion development method, and a paddle development method. The development conditions are, for example, 5 to 40° C. and about 1 to 10 minutes. After the resin film is developed with an alkaline developer, the resin film can be washed with water and dried.

Examples of the alkaline developer include an alkaline aqueous solution obtained by dissolving an alkaline compound such as sodium hydroxide, potassium hydroxide, aqueous ammonia, tetramethylammonium hydroxide, or choline in water to a concentration of 1 to 10 mass %. To the alkaline aqueous solution, for example, a water-soluble organic solvent such as methanol or ethanol, and for example, a surfactant can be added in appropriate amounts.

The shape of the pattern in the patterned resin film is not particularly limited as long as it has an irregularity structure, and examples thereof include a line-and-space pattern, a dot pattern, a hole pattern, and a lattice pattern.

### <Step (4)>

The method for producing a patterned resin film of the present invention can include, after the step (3), a step (4) of sufficiently curing the patterned resin film by a heat treatment (post-baking), as necessary, in order to sufficiently exhibit properties as an insulation film. The curing conditions are not particularly limited, and depending on the application of the patterned resin film, for example, heating is performed at a temperature of 100 to 350° C. for about 30 minutes to 10 hours.

### [Semiconductor Circuit Board]

By using the composition of the present invention, a semiconductor circuit board including the resin film (patterned resin film) having a pattern described above can be produced. Since the semiconductor circuit board includes a patterned resin film formed using the composition of the present invention described above, and preferably includes a patterned insulation film such as a surface protective film, an interlayer insulation film, or a planarization film, the semiconductor circuit board is useful as a highly reliable circuit board.

### EXAMPLES

Hereinafter, the present invention will be described more specifically based on Examples, but the present invention is not limited to these Examples. In the following description of Examples and others, unless otherwise specified, “part(s)” is used to mean “part(s) by mass”.

### <Synthesis of Polymer>

The weight average molecular weight (Mw) of a polymer obtained in the following synthesis example was measured by a gel permeation chromatography (GPC) method under the following conditions.
Column: product name “TSKgel α-M” (manufactured by TOSOH CORPORATION)
Solvent: N-methyl-2-pyrrolidone (NMP)
Temperature: 40° C.
Detection method: refractive index method
Standard substance: polystyrene
GPC apparatus: apparatus name “HLC-8320-GPC” manufactured by TOSOH CORPORATION

### [Synthesis Example 1] Synthesis of Polymer (A1)

In a 100 mL three-necked flask equipped with a reflux tube, 9.26 mmol of PPHT (manufactured by Nippon Fine Chemical Co., Ltd., the following formula (al-1)) as an acid dianhydride and 28.12 g of N-methyl-2-pyrrolidone (NMP) as a polymerization solvent were placed, and the mixture was stirred and dissolved under a nitrogen atmosphere.

To this solution, 5.00 mmol of 2,2-bis(3-amino-4-hydroxyphenyl)hexafluoropropane and 3.33 mmol of 1,3-bis(4-aminophenoxy)propane as diamines, and 1.85 mmol of 3-aminophenol as a terminal capping agent were added, and the mixture was stirred at 60° C. for 1 hour and then further stirred at 180° C. for 4 hours. The contents of the flask were cooled to room temperature, and then poured into 500 mL of distilled water to obtain a white solid. This solid was separated by filtration, washed 3 times with distilled water, and then vacuum-dried at 70° C. for 48 hours to obtain a polymer (A1) (polyimide). The obtained polymer (A1) was analyzed by, for example, ¹³C-NMR, and it was revealed that the polymer (A1) was a polymer having two types of structural units represented by the following formula (A1) and having a group represented by the following formula (Z1) at a polymer terminal. The obtained polymer (A1) was analyzed by, for example, ¹H-NMR, and it was found that the imidization rate was 100%. The weight average molecular weight (Mw) of the polymer (A1) was 24,700.

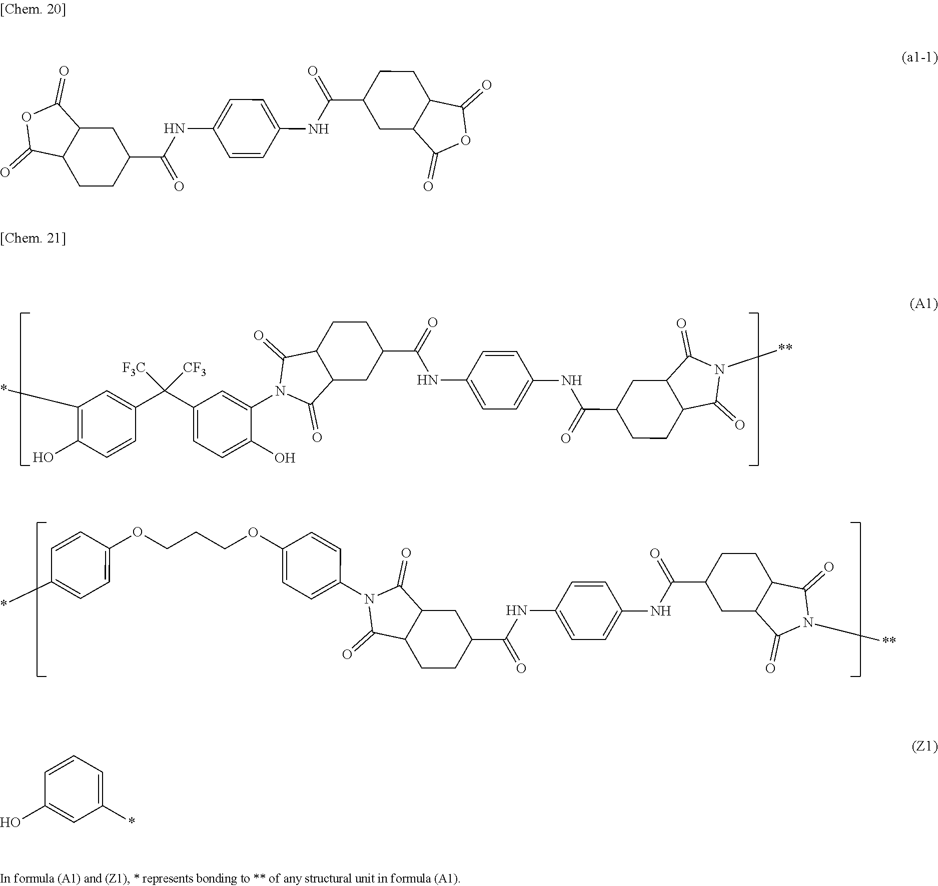

In formulae (A1) an and (Z1), * represents bonding to ** of any structural unit in formula (A1).

### [Synthesis Example 2] Synthesis of Polymer (A2)

In a 100 mL three-necked flask equipped with a reflux tube, 7.41 mmol of 4,4′-oxydiphthalic anhydride (ODPA) as an acid dianhydride, 10.00 g of methanol, and 0.5 mL of pyridine were placed, and the mixture was stirred under reflux for 5 hours under a nitrogen atmosphere. After the contents of the flask were cooled to room temperature, the contents were transferred to an eggplant flask, and methanol was distilled off with an evaporator to obtain a half methyl ester of ODPA.

Subsequently, in a 100 mL three-necked flask (flask A), the total amount of the obtained half methyl ester of ODPA and 20 g of NMP as a polymerization solvent were placed, and the mixture was stirred and dissolved under a nitrogen atmosphere, and cooled in an ice bath. After 15.56 mmol of thionyl chloride was added to this solution, the ice bath was removed, followed by stirring at 50° C. for 1 hour. To another 100 mL three-necked flask (flask B), 5.00 mmol of 2,2-bis(3-amino-4-hydroxyphenyl)hexafluoropropane and 1.67 mmol of 1,3-bis(4-aminophenoxy)propane as diamines and 1.48 mmol of 3-aminophenol as a terminal capping agent were added together with 15.00 g of NMP and 0.5 g of pyridine, and the mixture was stirred and dissolved under a nitrogen atmosphere, and cooled in an ice bath. The contents of the flask A cooled to room temperature were transferred to the flask B with a Teflon (registered trademark) tube and nitrogen, then the ice bath was removed, followed by stirring at room temperature for 3 hours. The contents of the flask B were poured into 500 mL of distilled water to obtain a white solid. This solid was separated by filtration, washed 3 times with distilled water, and then vacuum-dried at 70° C. for 48 hours to obtain a polymer (A2) (polyimide precursor). The obtained polymer (A2) was analyzed by, for example, ¹³C-NMR, and it was revealed that the polymer (A2) was a polymer having two types of structural units represented by the following formula (A2) and having a group represented by the following formula (Z1) at a polymer terminal. The obtained polymer (A2) was analyzed by, for example, ¹H-NMR, and it was found that the methyl esterification rate was 100%. The weight average molecular weight (Mw) of the polymer (A2) was 26,600.

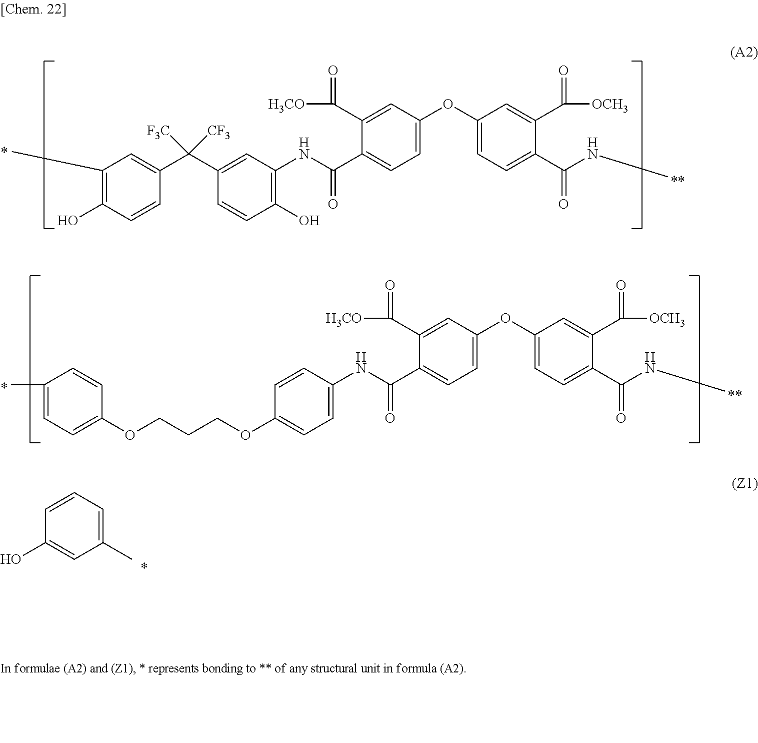

In formulae (A2) and (Z1), * represents bonding to ** of any structural unit in formula (A2).

### [Synthesis Example 3] Synthesis of Polymer (A3)

In 150 g of propylene glycol monomethyl ether, 62 g of p-t-butoxystyrene and 16 g of styrene were dissolved, the reaction temperature was maintained at 70° C. under a nitrogen atmosphere, and polymerization was allowed to proceed for 10 hours using 4 g of azoisobutyronitrile. Thereafter, sulfuric acid was added to the reaction solution, and a reaction was allowed to proceed for 10 hours while the reaction temperature was maintained at 90° C. to convert a p-t-butoxystyrene unit into a p-hydroxystyrene unit by deprotection. Ethyl acetate was added to the obtained copolymer, and washing with water was repeated five times, and the ethyl acetate layer was separated and the solvent was removed to obtain a polymer (A3) (p-hydroxystyrene/styrene copolymer). The obtained polymer (A3) was analyzed by, for example, 1H-NMR, and it was verified that the polymer (A3) had 70 mol % of a p-hydroxystyrene unit and 30 mol % of a styrene unit. The weight average molecular weight (Mw) of the polymer (A3) was 10,000.

### [Synthesis Example 4] Synthesis of Polymer (A4)

In a 100 mL three-necked flask equipped with a reflux tube, 9.26 mmol of a reaction product obtained by causing a reaction of 9.26 mmol of diphenyl ether-4,4′-dicarboxylic acid as a dicarboxylic acid derivative and 18.52 mmol of 1-hydroxy-1,2,3-benzotriazole, and 27.64 g of N-methyl-2-pyrrolidone as a polymerization solvent were placed, and the mixture was stirred and dissolved under a nitrogen atmosphere. Thereto, 5.00 mmol of 2,2-bis(3-amino-4-hydroxyphenyl)hexafluoropropane and 3.33 mmol of 1,3-bis(4-aminophenoxy)propane as diamines, and 1.85 mmol of 3-aminophenol as a terminal capping agent were added, and a reaction was allowed to proceed at 75° C. for 12 hours. After cooling to room temperature, the reaction solution was poured into 500 mL of distilled water to obtain a white solid. This solid was separated by filtration, washed 3 times with distilled water, and then vacuum-dried at 70° C. for 48 hours to obtain a polymer (A4) (polybenzoxazole precursor). The obtained polymer (A4) was analyzed by, for example, ¹³C-NMR, and it was revealed that the polymer (A4) was a polymer having two types of structural units represented by the following formula (A4) and having a group represented by the following formula (Z1) at a polymer terminal. The weight average molecular weight (Mw) of the polymer (A4) was 25,000.

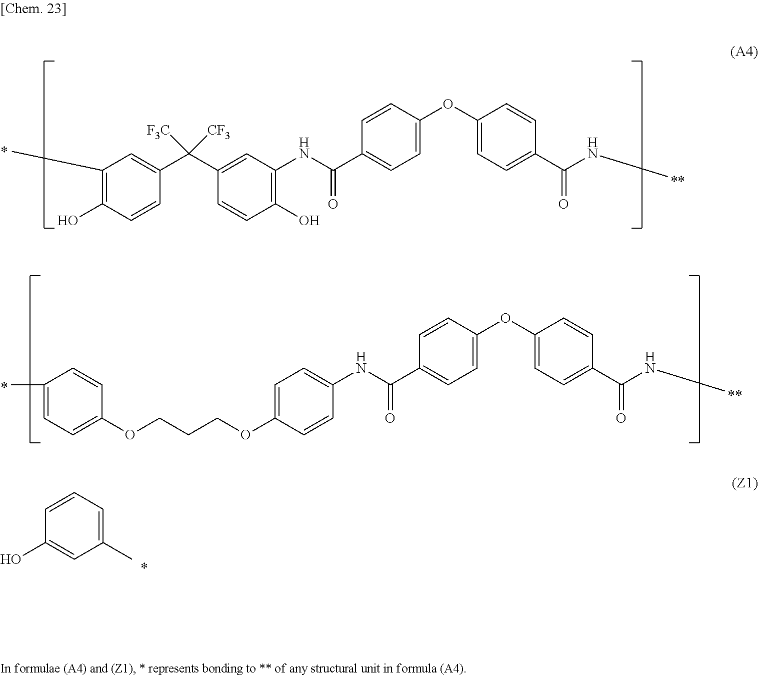

### [Synthesis Example 5] Synthesis of Polymer (A5)

In a 100 mL three-necked flask equipped with a reflux tube, 12.20 mmol of 2,2-bis(3-amino-4-hydroxyphenyl)hexafluoropropane and 9.98 mmol of 1,3-bis(4-aminophenoxy)propane as diamines and 61.4 g of γ-butyrolactone (GBL) as a polymerization solvent were placed, and the mixture was stirred and dissolved under a nitrogen atmosphere. Thereto, 17.75 mmol of PPHT was added as an acid dianhydride, and the mixture was stirred at 90° C. for 1 hour and then further stirred at 180° C. for 4 hours. After cooling to room temperature, the reaction solution was poured into 500 mL of distilled water to obtain a white solid. This solid was separated by filtration, washed 3 times with distilled water, and then vacuum-dried at 70° C. for 48 hours to obtain a polymer (A5) (polyimide). The obtained polymer (A5) was analyzed by, for example, ¹³C-NMR, and it was revealed that the polymer (A5) was a polymer having two types of structural units represented by the following formula (A5) and having a group represented by the following formula (Z2) at a polymer terminal. The obtained polymer (A5) was analyzed by, for example, 1H-NMR, and it was found that the imidization rate was 100%. The weight average molecular weight (Mw) of the polymer (A5) was 13,400.

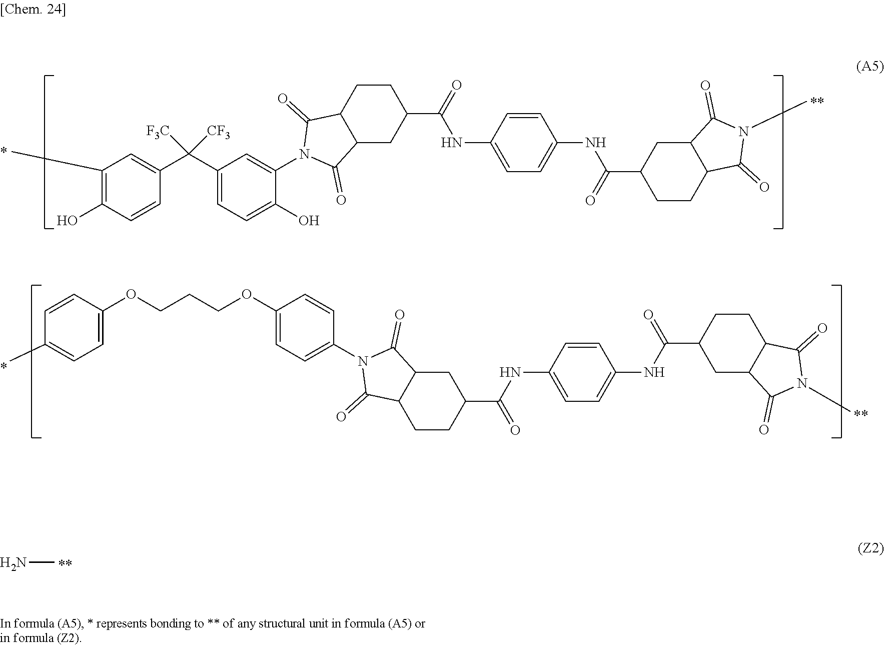

### [Synthesis Example 6] Synthesis of Polymer (A6)

To a reaction vessel, 5.5 mmol of sec-butyllithium as an initiator, 120 g of cyclohexane and 80 g of methylcyclohexane as polymerization solvents, and 111.3 mmol of tetrahydrofuran as a randomizer were added, and the temperature of the contents was set to −20° C. While dropwise addition was performed so that the temperature remains constant, 204.6 mmol of p-t-butoxystyrene was polymerized. Subsequently, the temperature of the contents was adjusted to −10° C., and then polymerization was allowed to proceed while 204.6 mmol of isoprene was added dropwise as a copolymerization monomer. The reaction was stopped with methanol to obtain a block copolymer of p-t-butoxystyrene/isoprene. The obtained polymer was subjected to a hydrogenation treatment in the presence of a Pd—C catalyst. Subsequently, a deprotection treatment was performed with sulfuric acid to obtain a polymer (A6) (block copolymer of p-hydroxystyrene/hydrogenated isoprene).

From analyses such as 1H-NMR and ¹³C-NMR of the obtained polymer (A6), the hydrogenation rate of isoprene moieties was 83%. The weight average molecular weight (Mw) of the obtained polymer (A6) was 9,100.

### [Synthesis Example 7] Synthesis of Polymer (A7)

In a reaction vessel, 89 g of p-t-butoxystyrene was dissolved in 150 g of propylene glycol monomethyl ether as a polymerization solvent, the reaction temperature was maintained at 70° C. under a nitrogen atmosphere, and polymerization was allowed to proceed for 10 hours using 4 g of azoisobutyronitrile as an initiator. Thereafter, sulfuric acid was added to the reaction solution, and a reaction was allowed to proceed for 10 hours while the reaction temperature was maintained at 90° C. to convert a p-t-butoxystyrene unit into a p-hydroxystyrene unit by deprotection. Ethyl acetate was added to the obtained polymer, and washing with water was repeated five times, and the ethyl acetate layer was separated and the solvent was removed to obtain a polymer (A7) (p-hydroxystyrene polymer).

From an analysis such as ¹H-NMR of the obtained polymer (A7), it was verified that the polymer (A7) had 100 mol % of a p-hydroxystyrene unit. The weight average molecular weight (Mw) of the obtained polymer (A7) was 10,000.

### [Synthesis Example 8] Synthesis of Polymer (A8)

In a 100 mL three-necked flask equipped with a reflux tube, 9.26 mmol of PPHT as an acid dianhydride and 28.12 g of NMP as a polymerization solvent were placed, and the mixture was stirred and dissolved under a nitrogen atmosphere. To this solution, 8.42 mmol of 1,3-bis(4-aminophenoxy)propane as a diamine, and 1.85 mmol of 3-aminophenol as a terminal capping agent were added, and the mixture was stirred at 60° C. for 1 hour and then further stirred at 180° C. for 4 hours. The contents of the flask were cooled to room temperature, and then poured into 500 mL of distilled water to obtain a white solid. This solid was separated by filtration, washed 3 times with distilled water, and then vacuum-dried at 70° C. for 48 hours to obtain a polymer (A8) (polyimide). The obtained polymer (A8) was analyzed by, for example, ¹³C-NMR, and it was revealed that the polymer (A8) was a polymer having a structural unit represented by the following formula (A8) and having a group represented by the following formula (Z1) at a polymer terminal. The obtained polymer (A8) was analyzed by, for example, ¹H-NMR, and it was found that the imidization rate was 100%. The weight average molecular weight (Mw) of the polymer (A8) was 14,300.

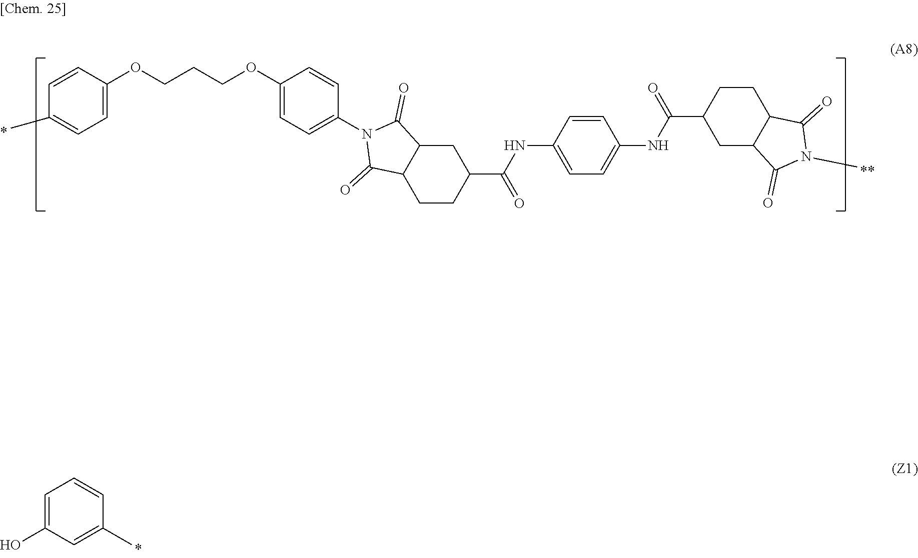

### [Synthesis Example 9] Synthesis of Polymer (A9)

In a 100 mL three-necked flask equipped with a reflux tube, 9.26 mmol of PPHT as an acid dianhydride and 28.12 g of NMP as a polymerization solvent were placed, and the mixture was stirred and dissolved under a nitrogen atmosphere. To this solution, 8.51 mmol of 4,4′-diaminodiphenyl ether as a diamine, and 1.85 mmol of 3-aminophenol as a terminal capping agent were added, and the mixture was stirred at 60° C. for 1 hour and then further stirred at 180° C. for 4 hours. The contents of the flask were cooled to room temperature, and then poured into 500 mL of distilled water to obtain a white solid. This solid was separated by filtration, washed 3 times with distilled water, and then vacuum-dried at 70° C. for 48 hours to obtain a polymer (A9) (polyimide). The obtained polymer (A9) was analyzed by, for example, ¹³C-NMR, and it was revealed that the polymer (A9) was a polymer having a structural unit represented by the following formula (A9) and having a group represented by the following formula (Z1) at a polymer terminal. The obtained polymer (A9) was analyzed by, for example, 1H-NMR, and it was found that the imidization rate was 100%. The weight average molecular weight (Mw) of the polymer (A9) was 18,800.

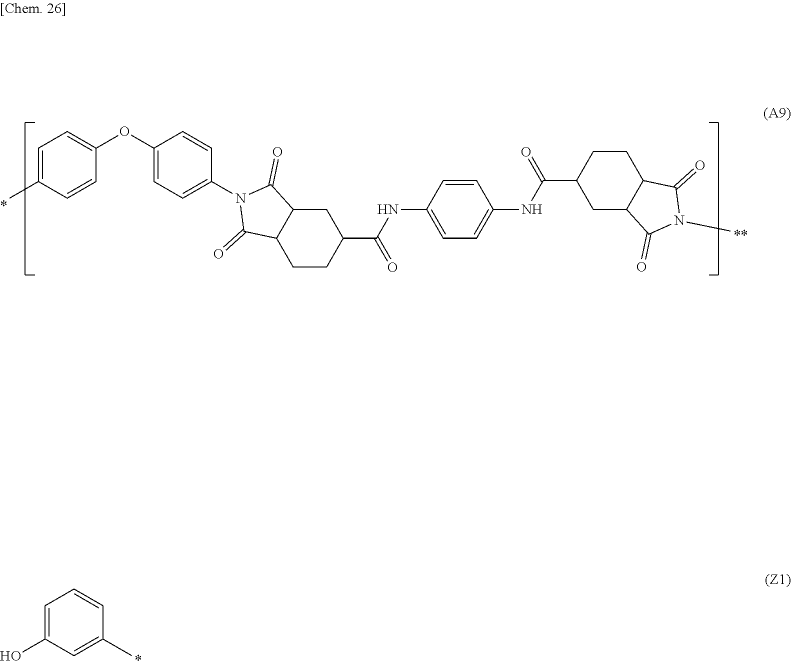

### [Synthesis Example 10] Synthesis of Polymer (A10)

In 150 g of propylene glycol monomethyl ether, 68 g of p-t-butoxystyrene and 10 g of styrene were dissolved, the reaction temperature was maintained at 70° C. under a nitrogen atmosphere, and polymerization was allowed to proceed for 10 hours using 4 g of azoisobutyronitrile. Thereafter, sulfuric acid was added to the reaction solution, and a reaction was allowed to proceed for 10 hours while the reaction temperature was maintained at 90° C. to convert a p-t-butoxystyrene unit into a p-hydroxystyrene unit by deprotection. Ethyl acetate was added to the obtained copolymer, and washing with water was repeated five times, and the ethyl acetate layer was separated and the solvent was removed to obtain a polymer (A10) (p-hydroxystyrene/styrene copolymer). The obtained polymer (A10) was analyzed by, for example, 1H-NMR, and it was verified that the polymer (A10) had 80 mol % of a p-hydroxystyrene unit and 20 mol % of a styrene unit. The weight average molecular weight (Mw) of the polymer (A10) was 11,000.

### [Synthesis Example 11] Synthesis of Compound (D1)

In a three-necked flask equipped with a reflux tube, 2.2 g of 2,4,6-trichloro-1,3,5-triazine and 36.0 g of methyl ethyl ketone were placed and dissolved under a nitrogen atmosphere. The contents of the flask were cooled to 0 to 5° C., then 4.3 g of 4-aminophenol was added thereto over 30 minutes, and then a sodium acetate aqueous solution (3.2 g of sodium acetate/18 g of water) as a base was added dropwise thereto over 30 minutes. Thereafter, heating and stirring were performed at 80° C. for 3 hours. The contents of the flask were poured into 300 g of cold water, and precipitates were collected and then recrystallized with hot water to obtain a compound (D1) represented by the following formula (D1). The structure of the obtained compound (D1) was checked by, for example, ¹H-NMR or ¹³C-NMR.

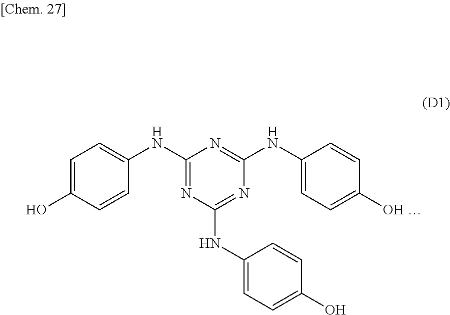 [SMILES: Oc1ccc(Nc2nc(Nc3ccc(O)cc3)nc(Nc3ccc(O)cc3)n2)cc1]

### [Synthesis Example 12] Synthesis of Compound (D2)

In a three-necked flask equipped with a reflux tube, 1.0 g of 2,4,6-trichloro-1,3,5-triazine and 16.4 g of methyl ethyl ketone were placed and dissolved under a nitrogen atmosphere. The contents of the flask were cooled to 0 to 5° C., then 0.51 g of aniline was added thereto over 30 minutes, and then a sodium acetate aqueous solution (1.33 g of sodium acetate/7.5 g of water) as a base was added dropwise thereto over 30 minutes. After stirring for 2 hours, 4.3 g of 4-aminophenol was added thereto over 30 minutes, and then the mixture was heated and stirred at 80° C. for 7 hours. The contents of the flask were poured into 300 g of cold water to collect precipitates, and then the precipitates were washed with n-hexane to obtain a compound (D2) represented by the following formula (D2). The structure of the obtained compound (D2) was checked by, for example, ¹H-NMR or 13C-NMR.

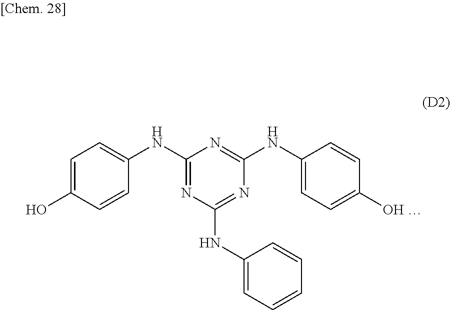 [SMILES: Oc1ccc(Nc2nc(Nc3ccccc3)nc(Nc3ccc(O)cc3)n2)cc1]

### [Synthesis Example 13] Synthesis of Compound (D3)

In a three-necked flask equipped with a reflux tube, 5.0 g of 2-amino-4,6-dichloropyrimidine, 7.0 g of 3-aminophenol, and 48 g of propylene glycol monomethyl ether were placed, and the mixture was stirred under reflux for 10 hours. The contents of the flask were allowed to cool to room temperature, neutralized with triethylamine, and then reprecipitated with water to obtain a compound (D3) represented by the following formula (D3). The structure of the obtained compound (D3) was checked by, for example, 1H-NMR or 13C-NMR.

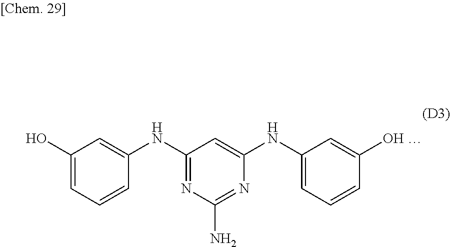 [SMILES: Nc1nc(Nc2cccc(O)c2)cc(Nc2cccc(O)c2)n1]

### [Synthesis Example 14] Synthesis of Compound (D4)

In a three-necked flask equipped with a reflux tube, 5.0 g of 6-chloro-2,4-diaminopyrimidine, 11.4 g of 3-aminophenol, and 100 g of N-methyl-2-pyrrolidone were placed, and the mixture was heated and stirred at 135° C. for 12 hours. The contents of the flask were allowed to cool to room temperature, then concentrated with an evaporator, and subjected to column purification (developing solvent: ethyl acetate/methanol=9/1) to remove the solvent to obtain a compound (D4) represented by the following formula (D4). The structure of the obtained compound (D4) was checked by, for example, ¹H-NMR or ¹³C-NMR.

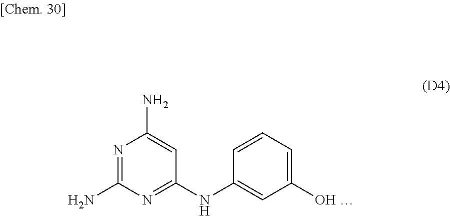 [SMILES: Nc1cc(Nc2cccc(O)c2)nc(N)n1]

### [Synthesis Example 15] Synthesis of Compound (D5)

A compound (D5) represented by the following formula (D5) was obtained in the same manner as in Synthesis Example 12 except that 0.78 g of 1-naphthylamine was used in place of 0.51 g of aniline. The structure of the obtained compound (D5) was checked by, for example, ¹H-NMR or ¹³C-NMR.

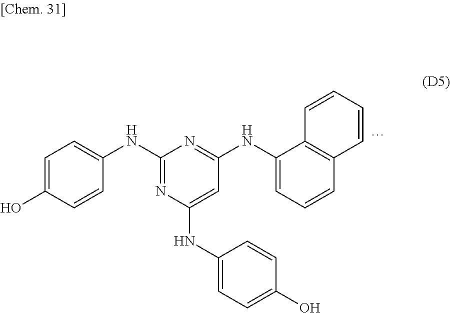 [SMILES: Oc1ccc(Nc2cc(Nc3cccc4ccccc34)nc(Nc3ccc(O)cc3)n2)cc1]

### [Synthesis Example 16] Synthesis of Compound (D6)

A compound (D6) represented by the following formula (D6) was obtained in the same manner as in Synthesis Example 12 except that 4.9 g of 4-amino-m-cresol was used in place of 4.3 g of 4-aminophenol. The structure of the obtained compound (D6) was checked by, for example, ¹H-NMR or 13C-NMR.

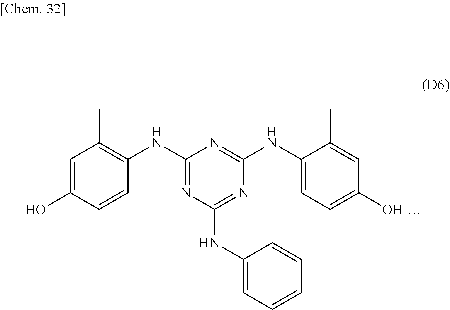 [SMILES: Cc1cc(O)ccc1Nc1nc(Nc2ccccc2)nc(Nc2ccc(O)cc2C)n1]

### [Synthesis Example 17] Synthesis of Compound (D7)

A compound (D7) represented by the following formula (D7) was obtained in the same manner as in Synthesis Example 13 except that 7.9 g of 4-amino-m-cresol was used in place of 7.0 g of 3-aminophenol. The structure of the obtained compound (D7) was checked by, for example, 1H-NMR or 13C-NMR.

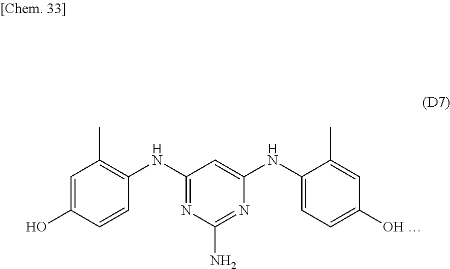 [SMILES: Cc1cc(O)ccc1Nc1cc(Nc2ccc(O)cc2C)nc(N)n1]

### [Synthesis Example 18] Synthesis of Compound (D8)

A compound (D8) represented by the following formula (D8) was obtained in the same manner as in Synthesis Example 14 except that 14.3 g of tyramine was used in place of 11.4 g of 3-aminophenol. The structure of the obtained compound (D8) was checked by, for example, ¹H-NMR or 13C-NMR.

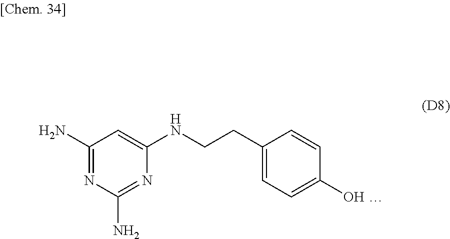 [SMILES: Nc1cc(NCCc2ccc(O)cc2)nc(N)n1]

### <Production of Photosensitive Resin Composition>

### EXAMPLES AND COMPARATIVE EXAMPLES

Photosensitive resin compositions of Examples 1 to 58 and Comparative Examples 1 to 9 were produced using the polymer (A), the photosensitizer (B), the crosslinking compound (C), the comparative compound (C′) of the compound (C), the compound (D), the comparative compound (D′) of the compound (D), the silane coupling agent, or the additive (F) shown in the following Table 1-1 and Table 1-7 (hereinafter also collectively referred to as “Table 1”) in the amounts (parts by mass) shown in Table 1, and also using the solvent (E) shown in Table 1. The obtained photosensitive resin compositions were evaluated as follows. The results are shown in Table 1.

### <Evaluation>

### <<Uniform Solubility>>

The dissolution in the solvent (E) was evaluated according to the following criteria.
◯: The prepared liquid was uniformly dissolved.
X: Some insoluble matters were observed in the prepared liquid, or the prepared liquid was cloudy.
   <<Cu adhesion>>

Each of the photosensitive resin compositions obtained in Examples 1 to 58 and Comparative Examples 1 to 9 was applied onto a Cu-sputtered Si substrate, then heated at 110° C. for 180 seconds, and then cured under the conditions shown in the following Table 1-1 or Table 1-2 to form a cured film having a film thickness of 10 μm. Subsequently, in accordance with the method described in JIS K 5600, cuts were made in a 100 square grid shape at intervals of 1 mm on the cured film using a cutter guide. A cellophane tape was strongly pressure-bonded to the grid portion of the cured film, and the end of the tape was peeled off at once at an angle of 45 degrees, and evaluation was performed according to the following criteria.
◯: None of the 100 squares was peeled off from the Cu substrate.
X: One or more of the 100 squares were peeled off from the Cu substrate.

### <<HAST Resistance>>

Each of the photosensitive resin compositions obtained in Examples 1 to 40 or Comparative Examples 1 to 9 was applied onto a Si substrate having a 500 nm-thick SiO₂ thin film formed on the surface and further having a comb-shaped electrode (thickness of electrode=4 μm, width of electrode=10 μm, distance between electrodes=10 μm) made of Cu thereon, followed by heating at 110° C. for 180 seconds, and then curing under the conditions shown in Tables 1-1 to 1-5 to form a cured film having a thickness of 10 μm. The substrate having the cured film was subjected to a highly accelerated stress test (HAST) for 96 hours using “AEI, EHS-221MD” manufactured by ESPEC Corporation under conditions of a temperature of 130° C., a humidity of 85%, and an applied voltage of 10 V, and evaluation was performed according to the following criteria.
◯: The resistance value did not fall below 10¹¹Ω during the test.
X: The resistance value fell below 10¹¹Ω during the test.

### <<Patterning Performance (Alkali Developability and Resolution)>>

Each of the photosensitive resin compositions obtained in Examples 41 to 58 was applied onto a 6-inch silicon wafer by a spin coating method, and then heated at 120° C. for 5 minutes using a hot plate to prepare a uniform resin film having a film thickness of 10 μm. Thereafter, exposure to an ultraviolet ray from a high-pressure mercury lamp was performed using an aligner (“MA-100” manufactured by SUSS MicroTec SE) through a mask in which many 2-μm-side square punched patterns were arranged such that the exposure dose at a wavelength of 350 nm was 8,000 J/m². Subsequently, immersion development was performed at 23° C. for a specified time using a 2.38 mass % tetramethylammonium hydroxide aqueous solution. Thereafter, the resulting product was washed with ultrapure water for 60 seconds and dried with air. The surface of the square (2 μm×2 μm) punched pattern thus obtained was observed at a magnification of 1,500 times using a scanning electron microscope (“S4200” manufactured by Hitachi, Ltd.) and evaluated according to the following criteria.

### (Alkali Developability)

◯: The squares of 2 μm×2 μm could be developed when the immersion time was less than 180 seconds.
Δ: The squares of 2 μm×2 μm could be developed when the immersion time was 180 seconds or more and less than 240 seconds.
X: The squares of 2 μm×2 μm could be developed when the immersion time was 240 seconds or more, or the squares of 2 μm×2 μm could not be developed even when the immersion time was 240 seconds or more.

### <<Resolution>>

◯: The squares of 2 μm×2 μm were resolved when the angle formed by the substrate and the pattern wall was 80 degrees or more.
Δ: The squares of 2 μm×2 μm were resolved when the angle formed by the substrate and the pattern wall was 60 degrees or more and less than 80 degrees.
X: Resolution was not achieved.

TABLE 1-1

| | | Exam- | Exam- | Exam- | Exam- | Exam- | Exam- | Exam- | Exam- | Exam- | Exam- |
|---|---|---|---|---|---|---|---|---|---|---|---|
| | | ple | ple | ple | ple | ple | ple | ple | ple | ple | ple |
| | | 1 | 2 | 3 | 4 | 28 | 29 | 30 | 31 | 5 | 6 |
| Polymer (A) | A1 | 100 | 100 | 100 | 100 | 100 | 100 | 100 | 100 | 100 | 100 |
| (parts by mass) | A2 | — | — | — | — | — | — | — | — | — | — |
| | A3 | — | — | — | — | — | — | — | — | — | — |
| | A4 | — | — | — | — | — | — | — | — | — | — |
| | A5 | — | — | — | — | — | — | — | — | — | — |
| | A6 | — | — | — | — | — | — | — | — | — | — |
| | A7 | — | — | — | — | — | — | — | — | — | — |
| Photosensitizer (B) | B1 | 25 | 25 | 25 | 25 | 25 | 25 | 25 | 25 | 25 | 25 |
| (parts by mass) | | | | | | | | | | | |
| Crosslinking compound (C) | C1 | 20 | 20 | 20 | 20 | 20 | 20 | 20 | 20 | 20 | 20 |
| (parts by mass) | C2 | — | — | — | — | — | — | — | — | — | — |
| | C3 | — | — | — | — | — | — | — | — | — | — |
| | C4 | — | — | — | — | — | — | — | — | — | — |
| | C5 | — | — | — | — | — | — | — | — | — | — |
| | C6 | — | — | — | — | — | — | — | — | — | — |
| Comparative compound (C′) | C′1 | — | — | — | — | — | — | — | — | — | — |
| (parts by mass) | | | | | | | | | | | |
| Compound (D) | D1 | 5 | — | — | — | — | — | — | — | — | — |
| (parts by mass) | D2 | — | 5 | — | — | — | — | — | — | 5 | 5 |
| | D3 | — | — | 5 | — | — | — | — | — | — | — |
| | D4 | — | — | — | 5 | — | — | — | — | — | — |
| | D5 | — | — | — | — | 5 | — | — | — | — | — |
| | D6 | — | — | — | — | — | 5 | — | — | — | — |
| | D7 | — | — | — | — | — | — | 5 | — | — | — |
| | D8 | — | — | — | — | — | — | — | 5 | — | — |
| Comparative compound (D′) | D′1 | — | — | — | — | — | — | — | — | — | — |
| (parts by mass) | D′2 | — | — | — | — | — | — | — | — | — | — |
| | D′3 | — | — | — | — | — | — | — | — | — | — |
| | D′4 | — | — | — | — | — | — | — | — | — | — |
| Silane coupling agent (parts | d3 | — | — | — | — | — | — | — | — | 3 | — |
| by mass) | d2 | — | — | — | — | — | — | — | — | — | 3 |
| Additive (F) | F1 | — | — | — | — | — | — | — | — | — | — |
| (parts by mass) | F2 | 0.1 | 0.1 | 0.1 | 0.1 | 0.1 | 0.1 | 0.1 | 0.1 | 0.1 | 0.1 |

| | | | | | | | | | | |
|---|---|---|---|---|---|---|---|---|---|---|
| Solvent (E) | E1 | E1 | E1 | E2 | E1 | E1 | E1 | E2 | E1 | E1 |
| Solid content concentration | 30 | 30 | 30 | 30 | 30 | 30 | 30 | 30 | 30 | 30 |
| (mass %) | | | | | | | | | | |
| Curing temperature (° C.) | 230 | 230 | 230 | 230 | 230 | 230 | 230 | 230 | 230 | 230 |
| Curing time (hour) | 1 | 1 | 1 | 1 | 1 | 1 | 1 | 1 | 1 | 1 |
| Uniform solubility | ◯ | ◯ | ◯ | ◯ | ◯ | ◯ | ◯ | ◯ | ◯ | ◯ |
| Cu adhesion | ◯ | ◯ | ◯ | ◯ | ◯ | ◯ | ◯ | ◯ | ◯ | ◯ |
| | 100/100 | 100/100 | 100/100 | 100/100 | 100/100 | 100/100 | 100/100 | 100/100 | 100/100 | 100/100 |
| HAST resistance | ◯ | ◯ | ◯ | ◯ | ◯ | ◯ | ◯ | ◯ | ◯ | ◯ |

TABLE 1-2

| | | Example | Example | Example | Example | Comparative | Comparative | Comparative | Comparative | Comparative |
|---|---|---|---|---|---|---|---|---|---|---|
| | | 7 | 8 | 9 | 38 | Example 1 | Example 2 | Example 3 | Example 4 | Example 9 |
| Polymer (A) | A1 | 100 | 100 | 100 | 100 | 100 | 100 | 100 | 100 | 100 |
| (parts by mass) | A2 | — | — | — | — | — | — | — | — | — |
| | A3 | — | — | — | — | — | — | — | — | — |
| | A4 | — | — | — | — | — | — | — | — | — |
| | A5 | — | — | — | — | — | — | — | — | — |
| | A6 | — | — | — | — | — | — | — | — | — |
| | A7 | — | — | — | — | — | — | — | — | — |
| Photosensitizer | B1 | 25 | 25 | 25 | 25 | 25 | 25 | 25 | 25 | 25 |
| (B) | | | | | | | | | | |
| (parts by mass) | | | | | | | | | | |
| Crosslinking | C1 | — | — | — | — | 20 | 20 | 20 | 20 | — |
| compound (C) | C2 | — | — | — | — | — | — | — | — | — |
| (parts by mass) | C3 | 20 | — | — | — | — | — | — | — | — |
| | C4 | — | 20 | — | — | — | — | — | — | — |
| | C5 | — | — | 20 | — | — | — | — | — | — |
| | C6 | — | — | — | 20 | — | — | — | — | — |
| Comparative | C′1 | — | — | — | — | — | — | — | — | 20 |
| compound (C′) | | | | | | | | | | |
| (parts by mass) | | | | | | | | | | |
| Compound (D) | D1 | — | — | — | — | — | — | — | — | — |
| (parts by mass) | D2 | 5 | 5 | 5 | 5 | — | — | — | — | 5 |
| | D3 | — | — | — | — | — | — | — | — | — |
| | D4 | — | — | — | — | — | — | — | — | — |
| | D5 | — | — | — | — | — | — | — | — | — |
| | D6 | — | — | — | — | — | — | — | — | — |
| | D7 | — | — | — | — | — | — | — | — | — |
| | D8 | — | — | — | — | — | — | — | — | — |
| Comparative | D′1 | — | — | — | — | — | — | 5 | — | — |
| compound (D′) | D′2 | — | — | — | — | — | — | — | — | — |
| (parts by mass) | D′3 | — | — | — | — | — | — | — | — | — |
| | D′4 | — | — | — | — | — | — | — | 5 | — |
| Silane coupling | d3 | | — | — | — | — | 3 | — | — | — |
| agent (parts by | d2 | | | | | | | | | |
| mass) | | | | | | | | | | |
| Additive (F) | F1 | — | — | — | — | — | — | — | 0.05 | — |
| (parts by mass) | F2 | 0.1 | 0.1 | 0.1 | 0.1 | 0.1 | 0.1 | 0.1 | — | 0.1 |

| | | | | | | | | | |
|---|---|---|---|---|---|---|---|---|---|
| Solvent (E) | E1 | E1 | E1 | E1 | E1 | E1 | E2 | E2 | E1 |
| Solid content | 30 | 30 | 30 | 30 | 30 | 30 | 30 | 37 | 30 |
| concentration | | | | | | | | | |
| (mass %) | | | | | | | | | |
| Curing temperature | 230 | 230 | 230 | 230 | 230 | 230 | 230 | 200 | 230 |
| (° C.) | | | | | | | | | |
| Curing time (hour) | 1 | 1 | 1 | 1 | 1 | 1 | 1 | 1 | 1 |
| Uniform solubility | ◯ | ◯ | ◯ | ◯ | ◯ | ◯ | ◯ | ◯ | ◯ |
| Cu adhesion | ◯ | ◯ | ◯ | ◯ | X | X | ◯ | X | X |
| | 100/100 | 100/100 | 100/100 | 100/100 | 0/100 | 35/100 | 100/100 | 0/100 | 12/100 |
| HAST resistance | ◯ | ◯ | ◯ | ◯ | X | ◯ | X | ◯ | X |

TABLE 1-3

| | | Example | Example | Example | Example | Example | Example | Example | Example | Example |
|---|---|---|---|---|---|---|---|---|---|---|
| | | 14 | 15 | 16 | 17 | 32 | 33 | 34 | 35 | 39 |
| Polymer (A) | A1 | — | — | — | — | — | — | — | — | — |
| (parts by mass) | A2 | — | — | — | — | — | — | — | — | — |
| | A3 | 100 | 100 | 100 | 100 | 100 | 100 | 100 | 100 | 100 |
| | A4 | — | — | — | — | — | — | — | — | — |
| | A5 | — | — | — | — | — | — | — | — | — |
| | A6 | — | — | — | — | — | — | — | — | — |
| | A7 | — | — | — | — | — | — | — | — | — |
| Photosensitizer (B) | B1 | 27.5 | 27.5 | 27.5 | 27.5 | 27.5 | 27.5 | 27.5 | 27.5 | 27.5 |
| (parts by mass) | | | | | | | | | | |
| Crosslinking compound (C) | C1 | — | — | — | — | — | — | — | — | — |
| (parts by mass) | C2 | 30 | 30 | 30 | 30 | 30 | 30 | 30 | 30 | 30 |
| | C3 | — | — | — | — | — | — | — | — | — |
| | C4 | — | — | — | — | — | — | — | — | — |
| | C5 | — | — | — | — | — | — | — | — | — |
| | C6 | — | — | — | — | — | — | — | — | — |
| Comparative compound (C′) | C′1 | — | — | — | — | — | — | — | — | — |
| (parts by mass) | | | | | | | | | | |
| Compound (D) | D1 | 5 | — | — | — | — | — | — | — | — |
| (parts by mass) | D2 | — | 5 | — | — | — | — | — | — | 2.5 |
| | D3 | — | — | 5 | — | — | — | — | — | 2.5 |
| | D4 | — | — | — | 5 | — | — | — | — | — |
| | D5 | — | — | — | — | 5 | — | — | — | — |
| | D6 | — | — | — | — | — | 5 | — | — | — |
| | D7 | — | — | — | — | — | — | 5 | — | — |
| | D8 | — | — | — | — | — | — | — | 5 | — |
| Comparative compound (D′) | D′1 | — | — | — | — | — | — | — | — | — |
| (parts by mass) | D′2 | — | — | — | — | — | — | — | — | — |
| | D′3 | — | — | — | — | — | — | — | — | — |
| | D′4 | — | — | — | — | — | — | — | — | — |
| Silane coupling agent (parts by | d3 | — | — | — | — | — | — | — | — | — |
| mass) | d2 | — | — | — | — | — | — | — | — | — |
| Additive (F) | F1 | 0.05 | 0.05 | 0.05 | 0.05 | 0.05 | 0.05 | 0.05 | 0.05 | 0.05 |
| (parts by mass) | F2 | — | — | — | — | — | — | — | — | — |

| | | | | | | | | | |
|---|---|---|---|---|---|---|---|---|---|
| Solvent (E) | E3 | E3 | E3 | E2 | E3 | E3 | E3 | E2 | E2 |
| Solid content concentration (mass %) | 37 | 37 | 37 | 37 | 37 | 37 | 37 | 37 | 37 |
| Curing temperature (° C.) | 200 | 200 | 200 | 200 | 200 | 200 | 200 | 200 | 200 |
| Curing time (hour) | 1 | 1 | 1 | 1 | 1 | 1 | 1 | 1 | 1 |
| Uniform solubility | ◯ | ◯ | ◯ | ◯ | ◯ | ◯ | ◯ | ◯ | ◯ |
| Cu adhesion | ◯ | ◯ | ◯ | ◯ | ◯ | ◯ | ◯ | ◯ | ◯ |
| | 100/100 | 100/100 | 100/100 | 100/100 | 100/100 | 100/100 | 100/100 | 100/100 | 100/100 |
| HAST resistance | ◯ | ◯ | ◯ | ◯ | ◯ | ◯ | ◯ | ◯ | ◯ |

TABLE 1-4

| | | Exam- | Exam- | Exam- | Exam- | Exam- | Exam- | | | | |
|---|---|---|---|---|---|---|---|---|---|---|---|
| | | ple | ple | ple | ple | ple | ple | Comparative | Comparative | Comparative | Comparative |
| | | 10 | 11 | 12 | 13 | 18 | 19 | Example 5 | Example 6 | Example 7 | Example 8 |
| Polymer (A) | A1 | — | — | — | — | — | — | — | — | — | — |
| (parts by mass) | A2 | 100 | 100 | 100 | 100 | — | — | — | — | — | — |
| | A3 | — | — | — | — | 100 | 100 | 100 | 100 | 100 | 100 |
| | A4 | — | — | — | — | — | — | — | — | — | — |
| | A5 | — | — | — | — | — | — | — | — | — | — |
| | A6 | — | — | — | — | — | — | — | — | — | — |
| | A7 | — | — | — | — | — | — | — | — | — | — |
| Photosensitizer | B1 | 25 | 25 | 25 | 25 | 27.5 | 27.5 | 27.5 | 27.5 | 27.5 | 27.5 |
| (B) | | | | | | | | | | | |
| (parts by mass) | | | | | | | | | | | |
| Crosslinking | C1 | 20 | 20 | 20 | 20 | — | — | — | — | — | — |
| compound (C) | C2 | — | — | — | — | 30 | 30 | 30 | 30 | 30 | 30 |
| (parts by mass) | C3 | — | — | — | — | — | — | — | — | — | — |
| | C4 | — | — | — | — | — | — | — | — | — | — |
| | C5 | — | — | — | — | — | — | — | — | — | — |
| | C6 | — | — | — | — | — | — | — | — | — | — |
| Comparative | C′1 | — | — | — | — | — | — | — | — | — | — |
| compound (C′) | | | | | | | | | | | |
| (parts by mass) | | | | | | | | | | | |
| Compound (D) | D1 | 5 | — | — | — | — | — | — | — | — | — |
| (parts by mass) | D2 | — | 5 | — | — | 5 | 5 | — | — | — | — |
| | D3 | — | — | 5 | — | — | — | — | — | — | — |
| | D4 | — | — | — | 5 | — | — | — | — | — | — |
| | D5 | — | — | — | — | — | — | — | — | — | — |
| | D6 | — | — | — | — | — | — | — | — | — | — |
| | D7 | — | — | — | — | — | — | — | — | — | — |
| | D8 | — | — | — | — | — | — | — | — | — | — |
| Comparative | D′1 | — | — | — | — | — | — | — | 5 | — | — |
| compound (D′) | D′2 | — | — | — | — | — | — | — | — | 5 | — |
| (parts by mass) | D′3 | — | — | — | — | — | — | — | — | — | 5 |
| | D′4 | — | — | — | — | — | — | — | — | — | — |
| Silane coupling | d3 | — | — | — | — | 3 | — | 3 | — | — | — |
| agent (parts by | d2 | — | — | — | — | — | 3 | — | — | — | — |
| mass) | | | | | | | | | | | |
| Additive (F) | F1 | 0.05 | 0.05 | 0.05 | 0.05 | 0.05 | 0.05 | 0.05 | 0.05 | 0.05 | 0.05 |
| (parts by mass) | F2 | — | — | — | — | — | — | — | — | — | — |

| | | | | | | | | | | |
|---|---|---|---|---|---|---|---|---|---|---|
| Solvent (E) | E1 | E1 | E1 | E2 | E3 | E3 | E3 | E2 | E2 | E2 |
| Solid content | 30 | 30 | 30 | 30 | 37 | 37 | 37 | 37 | 37 | 37 |
| concentration | | | | | | | | | | |
| (mass %) | | | | | | | | | | |
| Curing temperature | 300 | 300 | 300 | 300 | 200 | 200 | 200 | 200 | 200 | 200 |
| (° C.) | | | | | | | | | | |
| Curing time (hour) | 1 | 1 | 1 | 1 | 1 | 1 | 1 | 1 | 1 | 1 |
| Uniform solubility | ◯ | ◯ | ◯ | ◯ | ◯ | ◯ | ◯ | ◯ | ◯ | X |
| Cu adhesion | ◯ | ◯ | ◯ | ◯ | ◯ | ◯ | X | ◯ | ◯ | ◯ |
| | 100/100 | 100/100 | 100/100 | 100/100 | 100/100 | 100/100 | 10/100 | 100/100 | 100/100 | 100/100 |
| HAST resistance | ◯ | ◯ | ◯ | ◯ | ◯ | ◯ | ◯ | X | X | X |

TABLE 1-5

| | | Exam- | Exam- | Exam- | Exam- | Exam- | Exam- | Exam- | Exam- | Exam- | Exam- | Exam- |
|---|---|---|---|---|---|---|---|---|---|---|---|---|
| | | ple | ple | ple | ple | ple | ple | ple | ple | ple | ple | ple |
| | | 20 | 21 | 22 | 23 | 36 | 37 | 40 | 24 | 25 | 26 | 27 |
| Polymer (A) | A1 | — | — | — | — | — | — | — | — | — | — | — |
| (parts by mass) | A2 | — | — | — | — | — | — | — | — | — | — | — |
| | A3 | — | — | — | — | — | — | — | 50 | — | 50 | 75 |
| | A4 | 100 | 100 | — | — | — | — | — | — | — | — | — |
| | A5 | — | — | 100 | 100 | 100 | 100 | 100 | — | 75 | 50 | 25 |
| | A6 | — | — | — | — | — | — | — | 50 | — | — | |
| | A7 | — | — | — | — | — | — | — | — | 25 | — | — |
| Photosensitizer (B) | B1 | 25 | 25 | 25 | 25 | 25 | 25 | 25 | 25 | 35 | 27.5 | 27.5 |
| (parts by mass) | | | | | | | | | | | | |
| Crosslinking compound | C1 | 20 | 20 | 20 | 10 | 10 | 10 | 10 | — | 10 | — | — |
| (C) | C2 | — | — | — | 12 | 12 | 12 | 12 | 20 | 12 | 30 | 30 |
| (parts by mass) | C3 | — | — | — | — | — | — | — | — | — | — | — |
| | C4 | — | — | — | — | — | — | — | — | — | — | — |
| | C5 | — | — | — | — | — | — | — | — | — | — | — |
| | C6 | — | — | — | — | — | — | — | — | — | — | — |
| Comparative | C′1 | — | — | — | — | — | — | — | — | — | — | — |
| compound (C′) (parts | | | | | | | | | | | | |
| by mass) | | | | | | | | | | | | |
| Compound (D) | D1 | 5 | — | — | — | — | — | — | — | — | — | — |
| (parts by mass) | D2 | — | 5 | 5 | 5 | 2.5 | 10 | 2.5 | 3.5 | 5 | 5 | 5 |
| | D3 | — | — | — | — | — | — | 2.5 | — | — | — | — |
| | D4 | — | — | — | — | — | — | — | — | — | — | — |
| | D5 | — | — | — | — | — | — | — | — | — | — | — |
| | D6 | — | — | — | — | — | — | — | — | — | — | — |
| | D7 | — | — | — | — | — | — | — | — | — | — | — |
| | D8 | — | — | — | — | — | — | — | — | — | — | — |
| Comparative | D′1 | — | — | — | — | — | — | — | — | — | — | — |
| compound (D′) (parts | D′2 | — | — | — | — | — | — | — | — | — | — | — |
| by mass) | D′3 | — | — | — | — | — | — | — | — | — | — | — |
| | D′4 | — | — | — | — | — | — | — | — | — | — | — |
| Silane coupling agent | d3 | — | — | — | — | — | — | — | — | — | — | — |
| (parts by mass) | d2 | 3 | 3 | — | 5 | 5 | 5 | 5 | 3.5 | 5 | 3 | 3 |
| Additive (F) | F1 | — | — | — | — | — | — | — | 0.05 | — | 0.05 | 0.05 |
| (parts by mass) | F2 | 0.05 | 0.05 | 0.1 | 0.1 | 0.1 | 0.1 | 0.1 | — | 0.1 | — | — |

| | | | | | | | | | | | |
|---|---|---|---|---|---|---|---|---|---|---|---|
| Solvent (E) | E2 | E2 | E1 | E1 | E1 | E1 | E1 | E3 | E1 | E1 | E1 |
| Solid content concentration | 30 | 30 | 30 | 30 | 30 | 30 | 30 | 40 | 30 | 37 | 37 |
| (mass %) | | | | | | | | | | | |
| Curing temperature (° C.) | 300 | 300 | 230 | 230 | 230 | 230 | 230 | 200 | 230 | 230 | 230 |
| Curing time (hour) | 1 | 1 | 1 | 1 | 1 | 1 | 1 | 1 | 1 | 1 | 1 |
| Uniform solubility | ◯ | ◯ | ◯ | ◯ | ◯ | ◯ | ◯ | ◯ | ◯ | ◯ | ◯ |
| Cu adhesion | ◯ | ◯ | ◯ | ◯ | ◯ | ◯ | ◯ | ◯ | ◯ | ◯ | ◯ |
| | 100/100 | 100/100 | 100/100 | 100/100 | 100/100 | 100/100 | 100/100 | 100/100 | 100/100 | 100/100 | 100/100 |
| HAST resistance | ◯ | ◯ | ◯ | ◯ | ◯ | ◯ | ◯ | ◯ | ◯ | ◯ | ◯ |

TABLE 1-6

| | | Example | Example | Example | Example | Example | Example | Example | Example | Example |
|---|---|---|---|---|---|---|---|---|---|---|
| | | 41 | 42 | 43 | 44 | 45 | 46 | 47 | 48 | 49 |
| Polymer (A) | A8 | 90 | 80 | 70 | 60 | 50 | 40 | 30 | 20 | 10 |
| (parts by mass) | A9 | — | — | — | — | — | — | — | — | — |
| | A10 | 10 | 20 | 30 | 40 | 50 | 60 | 70 | 80 | 90 |
| Photosensitizer (B) | B1 | 27.5 | 27.5 | 27.5 | 27.5 | 27.5 | 27.5 | 27.5 | 27.5 | 27.5 |
| (parts by mass) | | | | | | | | | | |
| Crosslinking | C2 | 20 | 20 | 20 | 20 | 20 | 20 | 20 | 20 | 20 |
| compound (C) | | | | | | | | | | |
| (parts by mass) | | | | | | | | | | |
| Compound (D) | D2 | 5 | 5 | 5 | 5 | 5 | 5 | 5 | 5 | 5 |
| (parts by mass) | | | | | | | | | | |
| Additive (F) | F1 | 0.05 | 0.05 | 0.05 | 0.05 | 0.05 | 0.05 | 0.05 | 0.05 | 0.05 |
| (parts by mass) | | | | | | | | | | |
| Solvent (E) | | E1 | E1 | E1 | E1 | E1 | E1 | E1 | E1 | E1 |
| Solid content concentration (mass %) | | 37 | 37 | 37 | 37 | 37 | 37 | 37 | 37 | 37 |
| Curing temperature (° C.) | | 200 | 200 | 200 | 200 | 200 | 200 | 200 | 200 | 200 |
| Curing time (hour) | | 1 | 1 | 1 | 1 | 1 | 1 | 1 | 1 | 1 |
| Uniform solubility | | ◯ | ◯ | ◯ | ◯ | ◯ | ◯ | ◯ | ◯ | ◯ |
| Cu adhesion | | ◯ | ◯ | ◯ | ◯ | ◯ | ◯ | ◯ | ◯ | ◯ |
| | | 100/100 | 100/100 | 100/100 | 100/100 | 100/100 | 100/100 | 100/100 | 100/100 | 100/100 |
| Patterning | Alkali | Δ | ◯ | ◯ | ◯ | ◯ | ◯ | ◯ | ◯ | ◯ |
| | developability | | | | | | | | | |
| performance | Resolution | ◯ | ◯ | ◯ | ◯ | ◯ | ◯ | ◯ | ◯ | Δ |

TABLE 1-7

| | | Example | Example | Example | Example | Example | Example | Example | Example | Example |
|---|---|---|---|---|---|---|---|---|---|---|
| | | 50 | 51 | 52 | 53 | 54 | 55 | 56 | 57 | 58 |
| Polymer (A) | A8 | — | — | — | — | — | — | — | — | — |
| (parts by mass) | A9 | 90 | 80 | 70 | 60 | 50 | 40 | 30 | 20 | 10 |
| | A10 | 10 | 20 | 30 | 40 | 50 | 60 | 70 | 80 | 90 |
| Photosensitizer (B) | B1 | 25 | 25 | 27.5 | 27.5 | 27.5 | 27.5 | 27.5 | 27.5 | 25 |
| (parts by mass) | | | | | | | | | | |
| Crosslinking | C2 | 20 | 20 | 20 | 20 | 20 | 20 | 20 | 20 | 20 |
| compound (C) | | | | | | | | | | |
| (parts by mass) | | | | | | | | | | |
| Compound (D) | D2 | 5 | 5 | 5 | 5 | 5 | 5 | 5 | 5 | 5 |
| (parts by mass) | | | | | | | | | | |
| Additive (F) | F1 | 0.05 | 0.05 | 0.05 | 0.05 | 0.05 | 0.05 | 0.05 | 0.05 | 0.05 |
| (parts by mass) | | | | | | | | | | |
| Solvent (E) | | E1 | E1 | E1 | E1 | E1 | E1 | E1 | E1 | E1 |
| Solid content concentration (mass %) | | 37 | 37 | 37 | 37 | 37 | 37 | 37 | 37 | 37 |
| Curing temperature (° C.) | | 200 | 200 | 200 | 200 | 200 | 200 | 200 | 200 | 200 |
| Curing time (hour) | | 1 | 1 | 1 | 1 | 1 | 1 | 1 | 1 | 1 |
| Uniform solubility | | ◯ | ◯ | ◯ | ◯ | ◯ | ◯ | ◯ | ◯ | ◯ |
| Cu adhesion | | ◯ | ◯ | ◯ | ◯ | ◯ | ◯ | ◯ | ◯ | ◯ |
| | | 100/100 | 100/100 | 100/100 | 100/100 | 100/100 | 100/100 | 100/100 | 100/100 | 100/100 |
| Patterning | Alkali | Δ | ◯ | ◯ | ◯ | ◯ | ◯ | ◯ | ◯ | ◯ |
| | developability | | | | | | | | | |
| performance | Resolution | ◯ | ◯ | ◯ | ◯ | ◯ | ◯ | ◯ | ◯ | Δ |

The details of the photosensitizer (B), the crosslinking compound (C), the comparative compound (C′) of the compound (C), the comparative compound (D′) of the compound (D), the silane coupling agent, the solvent (E), and the additive (F) used in Table 1-1 to Table 1-7 are shown below.

### Photosensitizer (B)

(B1): Condensate of 4,4′-[1-[4-[1-(4-hydroxyphenyl)-1-methylethyl]phenyl]ethylidene]bisphenol and 1,2-naphthoquinonediazide-5-sulfonic acid (molar ratio=1.0 2.0)

### Crosslinking Compound (C) and Comparative Compound (C′)

(C1): TMOM-BP (3,3′,5,5′-tetrakis(methoxymethyl)-[1,1′-biphenyl]-4,4′-diol, manufactured by Honshu Chemical Industry Co., Ltd.)
(C2): HMOM-TPPA (4,4′-[1-[4-[1-[4-hydroxy-3,5-bis(methoxymethyl)phenyl]-1-methylethyl]phenyl]ethylidene]bis[2,6-bis(methoxymethyl)phenol], manufactured by Honshu Chemical Industry Co., Ltd.)
(C3): Compound represented by the following formula (C3)
(C4): Compound represented by the following formula (C4)
(C5): Compound represented by the following formula (C5)
(C6): Compound represented by the following formula (C6)
(C′1): Compound represented by the following formula (C′1)

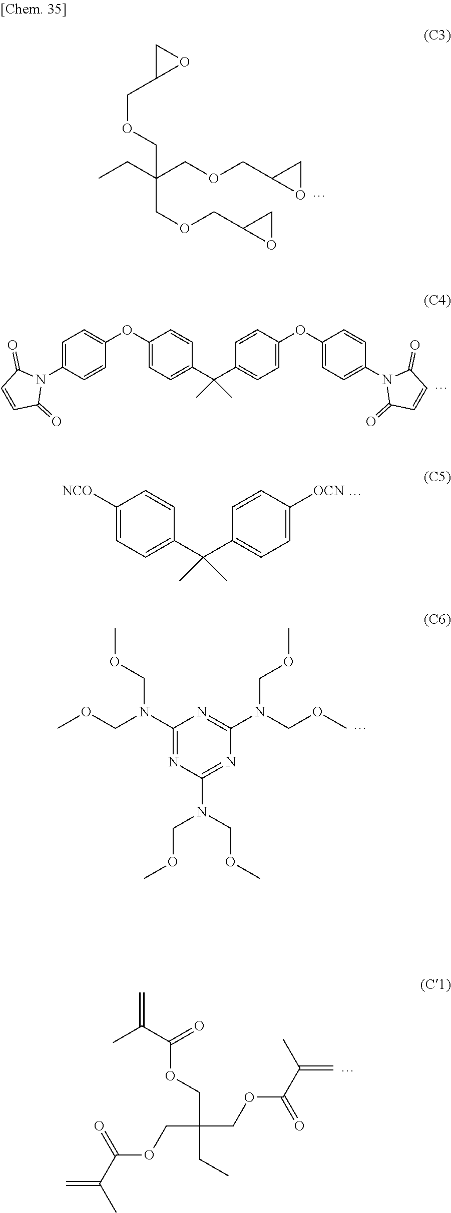

### Comparative Compound (D′) of Compound (D)

(D′1): Benzoguanamine
(D′2): 2,4-Diamino-6 methyl-1,3,5-triazine
(D′3): 1,3,5-Triazine-2,4,6-triamine
(D′4): Compound represented by the following formula (D′4), “ADK STAB LA-46” (manufactured by ADEKA CORPORATION)

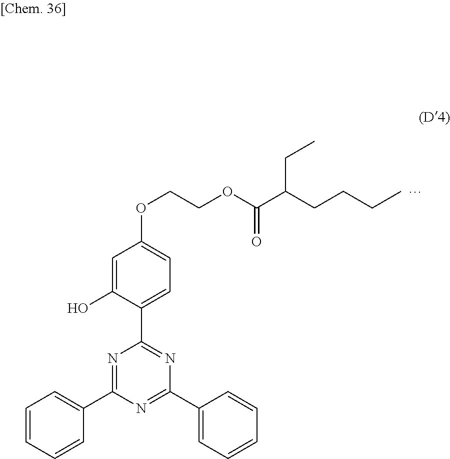 [SMILES: CCCCC(CC)C(=O)OCCOc1ccc(-c2nc(-c3ccccc3)nc(-c3ccccc3)n2)c(O)c1]

### Silane Coupling Agent

(d3): Tris(trimethoxysilylpropyl)isocyanurate
(d2): Trimethoxy[3-(phenylamino)propyl]silane

### Solvent (E)

(E1): γ-Butyrolactone (GBL)
(E2): N-methyl-2-pyrrolidone (NMP)
(E3): Ethyl lactate (EL)

### Additive (F)

(F1): Fluorine-based surfactant, trade name “NBX-15” (manufactured by Neos Corporation)
(F2): Siloxane-based surfactant, trade name “DOWSIL™ SH 8400 Fluid” (manufactured by Dow Chemical Japan Ltd.)

1: A photosensitive resin composition comprising:
(A) an alkali-soluble polymer;
(B) a photosensitizer;
(C) a crosslinking compound having a crosslinkable group that reacts with a component (D) below by heat;
(D) a compound represented by formula (1); and
(E) a solvent: 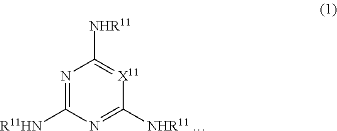
wherein, in the formula (1),
X¹¹ represents CR¹² or a nitrogen atom, wherein R¹² represents a hydrogen atom or a monovalent organic group,
three R¹¹'s independently represent a hydrogen atom or a group represented by formula (2), 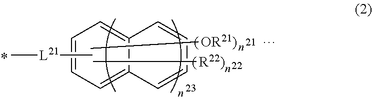
wherein, in the formula (2),
* represents a bond to a nitrogen atom in the formula (1),
L²¹ represents a single bond or an alkanediyl group having 1 to 5 carbon atoms,
R²¹ represents a hydrogen atom or a hydrocarbon group having 1 to 10 carbon atoms,
R²² represents a monovalent organic group,
n²¹ and n²² each independently represent an integer of 0 or more, and when n²² is an integer of 2 or more, a plurality of R²²'s each independently represent the monovalent organic group or represent a ring structure formed by combining the plurality of R²²'s together,
n²³ represents an integer of 0 or 1,
at least one of the three R¹'s is represented by formula (2), and in at least one group represented by formula (2), n²¹ is an integer of 1 or more, and at least one R²¹ is a hydrogen atom.

2: The photosensitive resin composition according to claim 1, wherein the photosensitizer (B) is a compound having a quinonediazide group.

3: The photosensitive resin composition according to claim 1, further comprising a silane coupling agent.

4: The photosensitive resin composition according to claim 1, wherein the alkali-soluble polymer (A) is at least one selected from the group consisting of a polyimide, a polyimide precursor, a polybenzoxazole, a polybenzoxazole precursor, a polymer comprising a structural unit (d) represented by formula (d), and a novolac resin: 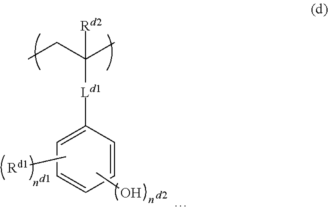
wherein, in the formula (d), L^{d1} represents a single bond, an ester bond, or an amide bond, R^{d1} represents an alkyl group having 1 to 5 carbon atoms or an alkoxy group having 1 to 5 carbon atoms, R^{d2} represents a hydrogen atom or an alkyl group having 1 to 5 carbon atoms, n^{d1} represents an integer of 0 to 4, and n^{d2} represents 1 or 2, provided that n^{d1}+n^{d2} is an integer of 5 or less.

5: The photosensitive resin composition according to claim 1, wherein in the formula (1), X¹¹ is a methine group (CH) or a nitrogen atom.

6: The photosensitive resin composition according to claim 1, wherein in the formula (1), two or more of the three R¹¹'s are each represented by the formula (2).

7: A method for producing a resin film having a pattern, the method comprising:
forming, on a substrate, a coating film of the photosensitive resin composition according to claim 1;
selectively exposing the coating film; and
developing the coating film after exposure with an alkaline developer.

8: A resin film having a pattern obtained by curing the photosensitive resin composition according to claim 1.

9: A semiconductor circuit board comprising the resin film having a pattern according to claim 8.
